(19) United States
(12) Patent Application Publication
(10) Pub. No.: US 2005/0169863 A1
(43) Pub. Date: Aug. 4, 2005

(54) 2-Thioacetamide compositions for stimulating the growth of keratin fibers and/or for reducing loss thereof

(75) Inventors: Roger Rozot, Lagny S/Marne (FR); Christophe Boulle, Lagny S/Marne (FR); Philippe Breton, Le Chesnay (FR)

Correspondence Address: BURNS DOANE SWECKER & MATHIS L L P, POST OFFICE BOX 1404, ALEXANDRIA, VA, 22313-1404, US

(73) Assignee: L'OREAL, Paris (FR)

(21) Appl. No.: 10/897,115

(22) Filed: Jul. 23, 2004

Related U.S. Application Data
(60) Provisional application No. 60/495,100, filed on Aug. 15, 2003.

(30) Foreign Application Priority Data
Jul. 31, 2003 (FR) 03/09476

(51) Int. Cl. A61K 7/09

(52) U.S. Cl. 424/70.5

(57) ABSTRACT

2-Thioacetamide compounds are suited for inducing and/or stimulating the growth of human keratin fibers, for example human hair, and/or reducing the loss and/or increasing the density thereof.

### CROSS-REFERENCE TO PRIORITY/PROVISIONAL APPLICATIONS

This application claims priority under 35 U.S.C. § 119 of FR 03/09476, filed Jul. 31, 2003, and of provisional application Ser. No. 60/495,100, filed Aug. 15, 2003, both hereby expressly incorporated by reference and both assigned to the assignee hereof. This application is also a continuation of said '100 provisional.

### BACKGROUND OF THE INVENTION

1. Technical Field of the Invention

The present invention relates to a care or makeup composition for human keratin fibers, containing a 2-thioacetamide compound, for inducing and/or stimulating the growth of keratin fibers and/or for reducing their loss. The invention also relates to a cosmetic treatment process for stimulating the growth of keratin fibers and/or for reducing their loss.

More especially, the invention relates to a care or makeup composition for the hair or the eyelashes, containing an effective amount of a 2-thioacetamide, for increasing their density and/or improving their appearance.

2. Description of Background and/or Related and/or Prior Art

Hair growth and hair renewal are mainly determined by the activity of the hair follicles and of their matrix environment. Their activity is cyclical and comprises essentially three phases, namely the anagenic phase, the catagenic phase and the telogenic phase.

The anagenic phase (active phase or growth phase), which lasts several years and during which the hair gets longer, is followed by a very short and transient catagenic phase that lasts a few weeks. During this phase, the hair undergoes a change, the follicle becomes atrophied and its dermal implantation appears higher and higher.

The terminal phase or telogenic phase, which lasts a few months, corresponds to a resting phase of the follicle and the hair ends up by falling out. At the end of this rest period, a new follicle is regenerated in situ and another cycle begins.

The head of hair is thus under permanent renewal, and, out of the approximately 150 000 hairs that make up a head of hair, about 10% are at rest and will be replaced within a few months.

The natural loss or falling-out of the hair may be estimated, on average, as being a few hundred hairs per day for a normal physiological state. This process of permanent physical renewal undergoes a natural change during aging, the hairs become finer and their cycles shorter.

In addition, various causes may result in a substantial, temporary or permanent loss of hair. This may be loss and impairment of hair at the terminal stage of pregnancy (post-partum), during states of dietary malnutrition or imbalance, or during states of asthenia or of hormonal dysfunction, as may be the case during or at the terminal stage of the menopause. It may also be a case of loss or impairment of the hair related to seasonal phenomena.

It may also be a matter of alopecia, which is essentially due to a disturbance in hair renewal, resulting, in a first stage, in acceleration of the frequency of the cycles to the detriment of the quality of the hair, and then of their quantity. The successive growth cycles result in hairs that are finer and finer and shorter and shorter, gradually transforming into an unpigmented down, thus resulting in a progressive impoverishment of the head of hair. Certain areas are preferentially affected, especially the temporal or frontal lobes in men, and a diffuse alopecia of the crown of the head in women.

The term alopecia also covers a whole family of afflictions of hair follicles whose final consequence is the permanent, partial or general loss of the hair. This is more particularly a matter of androgenic alopecia. In a large number of cases, early loss of hair occurs in genetically predisposed individuals; this is then a matter of androchronogenetic alopecia. This form of alopecia especially affects men.

It is moreover known that certain factors, such as hormonal imbalance, physiological stress or malnutrition, can accentuate the phenomenon.

In certain dermatoses of the scalp with an inflammatory component, for instance psoriasis or seborrhoeic dermatitis, hair loss may be greatly accentuated or may result in highly disrupted follicular cycles.

The cosmetics and pharmaceutical industries have for many years been investigating compositions for eliminating or reducing alopecia, and especially for inducing or stimulating hair growth or reducing its loss.

In this perspective, a large number of compositions comprising very diverse active agents have already been proposed, for instance 2,4-diamino-6-piperidinopyrimidine 3-oxide, or “minoxidil”, described in U.S. Pat. Nos. 4,139,619 and 4,596,812, or the numerous derivatives thereof such as those described, for example, in EP-0,353,123, EP-0,356,271, EP-0,408,442, EP-0,522,964, EP-0,420,707, EP-0,459,890 and EP-0,519,819.

Clinical studies have shown that PGF2-α analogues have the property of inducing the growth of body hairs and eyelashes in man and animals (Murray A. and Johnstone M. D., 1997, Am. J. Opht., 124(4), 544-547). In man, tests performed on the scalp have shown that a prostaglandin E2 analogue (viprostol) has the property of increasing the hair density (Roenigk H. H., 1988, Clinic Dermatol., 6(4), 119-121).

Moreover, WO 98/33497 describes pharmaceutical compositions containing prostaglandins or prostaglandin derivatives, for combating hair loss in man. Prostaglandins of the type A2, F2α and E2 are mentioned as being preferred.

However, prostaglandins are molecules with a very short biological half-life, which act in an autocrine or paracrine manner, this reflecting the local and labile nature of the metabolism of prostaglandins (Narumiya S. et al., 1999, Physiol. Rev., 79(4), 1193-1226).

It is thus seen to be important, in order to maintain and/or increase the hair density in man, to preserve the endogenous reserves of PGF2-α and similarly of PGE2 in various compartments of the hair follicle or its immediate cutaneous environment.

One solution that gives good results is the use of lipoxygenase-inhibiting compounds and/or cyclooxygenase-inducing compounds to promote hair growth; one theory is that the use of such compounds directs the metabolism of fatty acids towards the endogenous synthesis of prostaglandins in preference to other routes.

However, to further improve the results, it would be desirable to be able to prolong the activity of the prostaglandins involved in growing the hair and keeping it alive.

It is moreover well known that the programmes of differentiation of the keratinocytes of the epidermis and of the hair follicle are clearly different. Thus, it is known that the keratins of the hair shaft represent a family (Langbein et al., 2001, J. Biol. Chem. 276: 35123-35132) that is different from the one expressed in the epidermis, that differentiation markers such as the keratins K₁ and K₁₀ are not expressed in the hair follicle and in particular in the outer sheath (Lenoir et al., 1988, Dev. Biol. 130: 610-620), that trichohyalin (O'Guin et al., 1992, J. Invest. Dermatol. 98: 24-32) and keratin K6irs (Porter et al., 2001, Br. J. Dermatol. 145: 558-568) are expressed in the hair follicle, in particular in the inner sheath, but not in the epidermis, and that type-1 cyclooxygenase, although expressed in the epidermis, is not expressed in the keratinocytes of the hair follicle but in the dermal papilla (Michelet. et al., 1997, J. Invest. Dermatol. 108: 205-209).

### SUMMARY OF THE INVENTION

Surprisingly, it has now been demonstrated that an enzyme specifically involved in the degradation of these prostaglandins is present in the dermal papilla of the hair, which is a compartment that is a decisive factor in the life of the hair. Specifically, the presence of 15-hydroxyprostaglandin dehydrogenase (abbreviated as 15-PGDH) at this level has now been shown. It has also now been shown that the inhibition of 15-PGDH has a beneficial effect on hair growth.

Thus, the present invention features a care or treatment composition for keratin fibers, and especially human hair fibers, containing at least one particular inhibitor of 15-hydroxyprostaglandin dehydrogenase and a physiologically acceptable medium.

15-PGDH is a key enzyme in the deactivation of prostaglandins, in particular of PGF2-α and PGE2, which are important mediators of hair growth and survival. It corresponds to the classification EC 1.1.1.141 and is NAD⁺-dependent. It has been isolated from pig kidney; its inhibition with a thyroid hormone, triiodothyronine, at doses very much higher than the physiological doses, has especially been observed.

However, it has never been proposed to employ a 15-PGDH inhibitor to maintain and/or increase the density of human keratin fibers and especially the hair density and/or to reduce the heterogeneity of the diameters of the keratin fibers and especially of the hair in man. The expression “increase the density of human keratin fibers, and especially the hair density” means increasing the number of keratin fibers, and especially of hairs per cm² of skin or of scalp.

It has now been found that certain salified or non-salified 2-thioacetamide compounds are, surprisingly, endowed with activity that is favourable towards improving the density of human keratin fibers. It has moreover been found that these compounds are inhibitors of 15-hydroxyprostaglandin dehydrogenase.

The present invention thus features compositions for topical application, containing, in a physiologically acceptable medium, an effective amount of a 2-thioacetamide compound of formula (I), or a salt thereof: 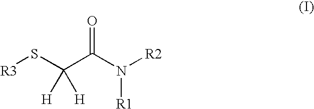
in which:
a) R₁ and R₂ independently represent:
1) a hydrogen atom,
2) a C₁-C₂₀ alkyl radical, optionally substituted with at least one substituent A₁,
3) a hydrocarbon-based ring C₁, optionally fused to a ring C₂ optionally containing at least one hetero atom to form a heterocycle Hy₁, these rings C₁ and C₂ optionally bearing at least one carbonyl or thiocarbonyl function and being substituted with at least one substituent A₃,
4) a heterocycle Hy₂, optionally fused to a ring C₂ optionally containing at least one hetero atom to form a heterocycle Hy₃, these rings Hy₂ and C₂ optionally bearing a carbonyl or thiocarbonyl function and being substituted with at least one substituent A₃, or
5) a group C(═NR)R′, C(═NR)NR′R″, COR, CSR, COOR or CONRR′, SO₂R or SO₂—NRR′;
b) R₃ represents:
1) a C₁-C₂₀ alkyl radical, optionally substituted with at least one substituent A₂,
2) a hydrocarbon-based ring C₃, optionally fused to a ring C₄ optionally containing at least one hetero atom to form a heterocycle Hy₃, these rings C₃ and C₄ optionally bearing at least one carbonyl or thiocarbonyl function and being substituted with at least one substituent A₃,
3) a heterocycle Hy₄ representing a pyrrole, furan, thiophene or pyrazole ring, optionally fused to a ring C₅ representing a phenyl, pyridine or pyrimidine ring, these two rings Hy₄ and C₅ optionally being substituted with at least one substituent A₃,
4) a pyridine ring optionally fused to a ring C₂ optionally containing at least one hetero atom to form a heterocycle Hy₁, these pyridine and C₂ rings optionally bearing at least one carbonyl or thiocarbonyl function and being substituted with at least one substituent A₄,
5) a heterocycle Hy₅ other than Hy₄ and the pyridine ring, optionally fused to a ring C₂ optionally containing at least one hetero atom to form a heterocycle Hy₁, these rings Hy₅ and C₂ optionally bearing at least one carbonyl or thiocarbonyl function and being substituted with at least one substituent A₃, or
6) a group C(═NR)R′, C(═NR)NR′R″, COR, CSR, COOR or CONRR′;
c) A₁ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a hydrocarbon-based ring C₆, optionally fused to a ring C₇ optionally containing at least one hetero atom to form a heterocycle Hy₆, these rings C₆ and C₇ optionally bearing at least one carbonyl or thiocarbonyl function and being substituted with at least one substituent A₅,
4) a heterocycle Hy₇ optionally fused to a ring C₇ optionally containing at least one hetero atom to form a heterocycle Hy₆, these rings Hy₇ and C₇ optionally bearing at least one carbonyl or thiocarbonyl function and being substituted with at least one substituent A₅;
d) A₂ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″, or
3) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
e) A₃ represents:
1) A₂, or
2) a C₁-C₂₀ alkyl group optionally substituted with at least one substituent A₅;
f) A4 represents:
1) a halogen,
2) a group CF₃, CN, OR, SR, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a C₁-C₂₀ alkyl group optionally substituted with at least one substituent A₅,
4) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
g)A₅ represents:
1) a halogen,
2) a group CF₃, CN, OR₅, SR₅, NR₅R₅, NR₅C(═NR₅′)NR₅′″R₅′″, COR₅, CSR₅, COOR₅, CONR₅R₅′, CSNR₅R₅′, NR₅CSRS₅′, NR₅CSNR₅′R₅″, NR₅COR₅′, NR₅CONR₅′R₅″, SO₂NR₅R₅′, NR₅SO₂R₅′, SO₂R₅ or SiR₅R₅′R₅″, R₅, R₅′, R₅″ being a hydrogen atom or a C₁-C₂₀ alkyl group,
3) a C₁-C₂₀ alkyl group, or
4) a ring C₁₀ optionally fused to another ring C₁₁, these rings optionally containing at least one hetero atom to form a heterocycle Hy₉ and/or bearing at least one carbonyl or thiocarbonyl function;
h) R, R′, R″ and R′″, which may be identical or different, represent:
1) a hydrogen atom,
2) a C₁-C₂₀ alkyl group optionally substituted with at least one substituent A₅, or
3) a ring C₁₂ optionally fused to another ring C₁₃, these rings optionally containing at least one hetero atom to form a heterocycle Hy₁₀ and being optionally substituted with at least one substituent A₅;
i) Hy₁ to Hy₁₀ independently represent a heterocycle optionally containing from 1 to 4 hetero atoms selected from among N, O and S.

This invention also features the use, especially the cosmetic use (regime or regimen), of at least one 2-thioacetamide compound of formula (I), or a salt thereof, as defined above, as an agent for inducing and/or stimulating the growth of human keratin fibers, especially the eyelashes and the hair, and/or for reducing their loss and/or increasing their density.

The human keratin fibers to which the invention applies are especially head hair, the eyebrows, the eyelashes, beard hair, moustache hair and pubic hair. The invention relates more especially to human head hair and/or eyelashes.

The invention also features the cosmetic use of at least one 2-thioacetamide compound of formula (I), or a salt thereof, in a human cosmetic haircare composition for reducing hair loss and/or for increasing its density and/or for treating androchronogenetic alopecia. This invention also features the use of at least one 2-thioacetamide compound of formula (I), or a salt thereof, for the preparation of a human hair composition, which is intended to induce and/or stimulate hair growth and/or reduce its loss and/or increase its density and/or treat androgenic alopecia.

The present invention also relates to the cosmetic use of at least one 2-thioacetamide compound of formula (I), or a salt thereof, in a human cosmetic haircare composition for treating alopecia of natural origin and in particular androgenic alopecia, and also to the use of at least one 2-thioacetamide compound of formula (I), or a salt thereof, for the preparation of a human haircare composition for treating alopecia of natural origin and in particular androgenic alopecia. Thus, this composition makes it possible to keep the head of hair in good condition and/or to combat natural hair loss, more particularly of humans.

Too, this invention also features the cosmetic use of at least one 2-thioacetamide compound of formula (I), or a salt thereof, in a cosmetic care and/or makeup composition for human eyelashes, to induce and/or stimulate the growth of the eyelashes and/or to increase their density, and also the use of at least one 2-thioacetamide compound of formula (I), or a salt thereof, for the preparation of a care and/or treatment composition for human eyelashes, which is intended to induce and/or stimulate the growth of the eyelashes and/or to increase their density. This composition thus makes it possible to keep the eyelashes in good condition and/or to improve their condition and/or appearance.

And this invention also features the use of at least one 2-thioacetamide compound of formula (I), or a salt thereof, as a 15-hydroxyprostaglandin dehydrogenase inhibitor. The invention also relates to the use of at least one 2-thioacetamide compound of formula (I), or a salt thereof, for the manufacture of a composition for treating disorders linked to the presence of 15-hydroxyprostaglandin dehydrogenase in man.

### DETAILED DESCRIPTION OF BEST MODE AND SPECIFIC/PREFERRED EMBODIMENTS OF THE INVENTION

The term “15-hydroxyprostaglandin dehydrogenase inhibitor” means a compound of formula (I) that is capable of inhibiting or reducing the activity of the enzyme 15-PGDH, and/or capable of inhibiting, reducing or slowing down the reaction catalysed by this enzyme.

According to one advantageous embodiment of the invention, the compound of formula (I) is a specific inhibitor of 15-PGDH; the term “specific inhibitor” means an active agent that has little or no inhibitory effect on the synthesis of prostaglandins, in particular on the synthesis of PGF2-α or PGE2. According to one particular embodiment of the invention, the 15-PGDH inhibitor has little or no inhibitory effect on prostaglandin synthase (PGF synthase).

Specifically, it has now been found that PGF synthase is also expressed in the dermal papilla. Maintaining an effective amount of prostaglandins at the site of action thus results from a complex biological equilibrium between the synthesis and degradation of these prostaglandins. The exogenous supply of compounds that inhibit catabolism will therefore be less effective if this activity is combined with an inhibition of the synthesis of these prostaglandins.

Advantageously, the compounds of formula (I), in salified or non-salified form, show inhibitory activity on 15-PGDH that is higher than the inhibitory activity on PGF synthase. In particular, the ratio between the inhibitory activities on PGF synthase and on 15-PGDH, respectively, for a given concentration, determined especially by the concentrations that inhibit 50% of the enzymatic activity, respectively, of PGF synthase (IC_{50fs}) and of 15-PGDH (IC_{50dh}), is at least greater than 1, especially at least 3:1 and advantageously greater than or equal to 5:1. The preferred compounds of the invention have an IC_{50fs}/IC_{50dh} ratio of greater than or equal to 10:1 and in particular greater than or equal to 15.

In the text hereinbelow, and unless specifically mentioned, the use of the term “compound of formula (I)” should be understood as meaning not only the compound of formula (I) in acidic or basic form but also a salt thereof. It may also be in a tautomeric form.

According to the invention, the term “at least one” means one or more (2, 3 or more). In particular, the composition may contain one or more compounds of formula (I). This or these compound(s) may be cis or trans isomers, or a mixture of cis/trans isomers. This or these compound(s) may also be in tautomeric form. This or these compound(s) may also be enantiomers and/or diastereoisomers or a mixture of these isomers, in particular a racemic mixture.

For the purposes of the invention, the term “hydrocarbon-based” refers to a group of hydrogen and carbon atoms.

For the purposes of the invention, the term “alkyl radical” means a hydrocarbon-based radical, which may be linear or branched and saturated or unsaturated. In particular, the alkyl radical contains from 1 to 20 and preferably from 1 to 10 carbon atoms. As examples of alkyl radicals that may be used in the invention, mention may be made of methyl, ethyl, isopropyl, n-butyl, tert-butyl, n-hexyl, 2-ethylhexyl, ethylene and propylene radicals.

According to the invention, the rings C₁ to C₁₃, Hy₂, Hy₄ and Hy₅ of formula (I) contain from 3 to 7 atoms and better still 5 or 6 atoms, and may be saturated or unsaturated. In addition, the rings C₂, C₄, C₇, C₈, C₉, C₁₀, C₁₁, C₁₂ and C₁₃ may optionally comprise one or more hetero atoms such as S, N or O or combinations thereof, and may thus form heterocycles. Hy₇ represents a heterocycle optionally comprising from 1 to 4 hetero atoms chosen from N, S and O and a combination thereof and containing from 3 to 15 atoms and better still 5 or 6 atoms. According to one particular embodiment of the invention, the heterocycle Hy₇ may contain up to 15 atoms, for instance a crown ether containing 5-CH₂CH₂O— units. These rings may also be fused to another ring of identical or different chemical nature. In addition, these rings may be substituted, in particular with a substituent A₃ or A₅.

As saturated hydrocarbon-based rings that may be used in the invention, mention may be made of cyclopropyl, cyclobutyl, cyclopentyl, cyclohexyl and cycloheptyl radicals. Unsaturated hydrocarbon-based rings that may be mentioned include cyclohexenyl and phenyl rings. As fused hydrocarbon-based rings that may be used, mention may be made of the naphthyl radical.

According to the invention, R₁ and/or R₂ may represent a hydrocarbon-based ring as defined above, in particular a saturated ring. According to the invention, R₁ and/or R₂ may also represent a heterocycle Hy₂ containing from 3 to 7 and better still 5 or 6 atoms, and comprising from 1 to 4 hetero atoms chosen from N, O and S, and a combination thereof. This heterocycle may also bear one or more carbonyl or thiocarbonyl functions. By way of example, Hy₂ represents one of the following heterocycles: azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazolidine, dihydopyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, diazepine. Preferably, Hy₂ represents a piperidine ring. This heterocycle Hy₂ may also be fused to a saturated or unsaturated ring C₂ of 4 to 7 atoms, C₂ optionally containing at least one hetero atom chosen from O, N and S to form a heterocycle Hy₁. C₂ may also bear one or more carbonyl or thiocarbonyl functions. As examples of heterocycles Hy, that may be used in the invention, mention may be made of pyridine, piperidine, morpholine, pyrrole, furan, thiophene, imidazole, oxazole, thiazole, pyrazole, pyrimidine, piperazine, pyrazine, pyridazine, triazine, pyrrolidine and thiazolidine rings.

According to the invention, R₁ and/or R₂ may also represent an alkyl radical, at least one of the hydrogens of which is replaced with a heterocycle Hy₇. This heterocycle may contain from 3 to 7 atoms and better still 5 or 6 atoms, and may comprise from 1 to 4 hetero atoms chosen from N, O and S, and a combination thereof. According to one particular embodiment of the invention, this heterocycle Hy₇ may contain up to 15 atoms, for instance a crown ether containing 5-CH₂CH₂O— units. According to another embodiment, Hy₇ bears a carbonyl function. As hetero-cycles Hy₇ that may be used in the invention, mention may be made of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, pyran, dihydropyran, tetrahydropyran, pyrimidine, dihydropyrimidine, tetrahydropyrimidine, piperazine, pyridazine, pyrazine, triazine, morpholine, azepine and diazepine rings, crown ether rings containing 15 atoms and 5-CH₂CH₂O— units, and pyrrolidinone rings. Preferably, Hy₇ represents one of the following rings: pyrrole, oxazole, pyridine, furan, pyrrolidine, morpholine, tetrahydrofuran, crown ether containing 15 atoms (5-CH₂CH₂O units) or pyrrolidinone.

According to the invention, R₃ may represent a saturated or unsaturated hydrocarbon-based ring C₃ of 3 to 7 carbon atoms and better still 5 or 6 carbon atoms, optionally fused to a heterocycle Hy₃. This heterocycle Hy₃ may contain from 4 to 7 atoms and better still 5 or 6 atoms and may comprise from 1 to 4 hetero atoms chosen from N, O and S, and a combination thereof. In addition, this heterocycle Hy₃ may bear one or more carbonyl or thiocarbonyl finctions.

As heterocycles Hy₃ that may be used in the invention, mention may be made of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine rings. Preferably, Hy₃ represents one of the following rings: pyrrole, pyridine, pyrimidine, imidazole, triazole, furan, thiophene, oxazole, thiazole.

According to the invention, R₃ may also represent:
a pyridine ring optionally fused to a ring C₂ of identical or different chemical nature,
a heterocycle Hy₄ chosen from pyrrole, furan, thiophene and pyrazole rings, optionally fused to a phenyl, pyridine or pyrimidine ring,
another heterocycle Hy₅ containing from 3 to 7 atoms and better still 5 or 6 atoms, and from 1 to 4 hetero atoms chosen from N, O and S, and a combination thereof. This heterocycle Hy₅ may also bear one or more carbonyl or thiocarbonyl functions and may be fused to another ring C₂ of identical or different chemical nature.

By way of example, the heterocycle Hy₅ is chosen from azetidine, dihydropyrrole, pyrrolidine, dihydrofuran, tetrahydrofuran, dihydrothiophene, tetrahydrothiophene, imidazole, dihydroimidazole, imidazolidine, thiazole, dihydrothiazole, thiazolidine, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, diazepine, 2-benzothiazolyl and thiazolo[2,3-c][1,2,4]triazole rings. Preferably, Hy₅ represents one of the following rings: 2-benzothiazolyl, thiazolo[2,3-c][1,2,4]triazole, imidazole, thiazole, triazole, oxazole, pyrimidine, pyrazine and pyridazine.

As examples of heterocycles Hy₁, Hy₆, Hy₈, Hy₉ and Hy₁₀ that may be used in the invention, mention may be made, independently, of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine rings. Preferably, pyrrole, pyrrolidine, imidazole, furan, pyrazole, pyridine, pyrimidine, triazole, pyrazine, pyridazine, piperidine, piperazine or morpholine rings are used.

The heterocycles of formula (I) may also be fused to a ring of identical or different chemical nature.

As fused heterocycles that may be used in the invention, mention may be made of purine and pteridine rings. As heterocycles fused to a hydrocarbon-based ring, which may be used in the invention, mention may be made of benzofuran, benzothiophene, benzothiazole, indole, benzimidazole, quinoline, isoquinoline and quinazoline rings.

As halogen atoms that may be used in the invention, mention may be made of chlorine, fluorine or bromine atoms, and better still fluorine and chlorine atoms.

According to the invention, the compounds of formula (I) are in isolated form, i.e., in non-polymeric form.

According to one particular embodiment of the invention, at least one from among R₁ and R₂ represents a hydrogen atom; a C₁-C₂₀ alkyl radical, at least one hydrogen of which is replaced with at least one substituent Al; a saturated or unsaturated hydrocarbon-based radical of 3 to 6 carbon atoms, at least one hydrogen of which is optionally substituted with a halogenated group such as CF₃ or halogen atoms and especially with 1 or 2 chlorine or bromine atoms when the ring is a phenyl. These groups and halogen atoms may be located in the para, meta or ortho position relative to the nitrogen atom bearing R₁ and R₂. The hydrocarbon-based ring is especially a cyclopentyl, cyclopropyl, cyclobutyl or cyclohexyl radical or an aryl radical, for instance phenyl. In particular, R₁ represents H and R₂ represents a cyclopentyl, cyclopropyl, cyclobutyl or cyclohexyl radical or a phenyl radical substituted with a bromine atom or 2 chlorine atoms.

According to another embodiment, R₁ represents H and R₂ represents a saturated or unsaturated heterocycle of 5 or 6 atoms, containing from 1 to 4 heteroatoms chosen from N, O and S and optionally substituted with at least one alkyl, CF₃, OR, SR, NRR′, COR, COOR, CONRR′ or NRCOR′ radical with R and R′ representing H or alkyl. For example, R₂ represents a piperidine ring optionally substituted with at least one C₁-C₁₀ alkyl radical, and especially methyl.

According to another embodiment, R₁ represents H and R₂ represents a saturated C₁-C₂₀ alkyl, optionally substituted with at least one substituent A₁. In particular, A₁ represents a saturated or unsaturated ring of 5 or 6 atoms, optionally containing from 1 to 4 hetero atoms chosen from N, O and S, which may contain at least one carbonyl function and may be optionally substituted with at least one group chosen from alkyl, F, CF₃, OR, SR, NRR′, COR, COOR, CONRR′ and NRCOR′ with R and R′ representing H or alkyl; a crown ether ring containing 15 atoms and 5-CH₂CH₂O— units; a group chosen from CF₃, OR, SR, NRR′, COR, COOR, CONRR′, NRCOR′ and SiRR′R″ with R, R′ and R″ representing H or alkyl.

According to another embodiment of the invention, R₃ represents a phenyl or a heterocycle of 5 or 6 atoms, substituted with 1 or 2 substituents A₃ or fused to a hydrocarbon-based or heterocyclic ring of 5 or 6 atoms. In particular, R₃ represents a heterocycle comprising as hetero atom at least one nitrogen atom and optionally a sulfur atom, this heterocycle being optionally fused to a phenyl or thiazole ring, which is itself optionally fused to a hydrocarbon-based ring, especially phenyl, or substituted with a group COR or CN or two groups COR and CN, respectively. In particular, R₃ represents a phenyl, 2-benzothiazolyl, 2-pyridyl, 6-acetonicotinonitrile, triazolyl or 4a,8a-dihydrobenzo[4,5]thiazolo[2,3-c][1,2,4]triazolyl group.

According to one particular embodiment of the invention, the 2-thioacetamide compound has one of the formulae (II) and (III) below: 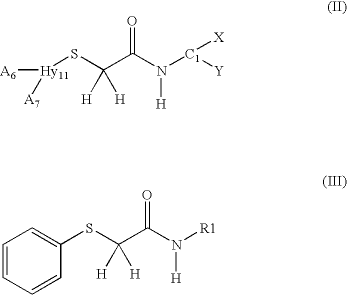
in which X and Y independently represent a hydrogen atom or a halogen atom; C₁ represents a saturated or unsaturated hydrocarbon-based ring of 3 to 6 carbon atoms; Hy₁₁ represents a heterocycle of 5 or 6 atoms containing at least one hetero atom chosen from N and S, and a combination thereof; A₆ and A₇ independently represent a substituent chosen from hydrogen, alkyl, COR, OR, SR, CN, COOR and saturated or unsaturated rings C₈ of 5 or 6 atoms, optionally comprising from 1 to 4 hetero atoms chosen from S and N, and combinations thereof, and/or being optionally fused to a hydrocarbon-based ring C₉ of 5 or 6 carbon atoms; R₁ represents a linear or branched, saturated or unsaturated C₁-C₂₀ alkyl radical, optionally substituted with at least one substituent A₁, a hydrocarbon-based ring or a heterocycle Hy₂ optionally substituted with at least one substituent A₃, C₈, C₉, A₁, Hy₂ and A₃ have the meaning given above.

According to the invention, the expression “salts of a compound of formula (I)” means the organic or mineral salts of a compound of formula (I).

As mineral salts that may be used according to the invention, mention may be made of the sodium or potassium salts, and also the zinc (Zn²⁺), calcium (Ca²⁺), copper (Cu²⁺), iron (Fe²⁺), strontium (Sr²⁺), magnesium (Mg²⁺) and manganese (Mn²⁺) salts; hydroxides and carbonates.

The organic salts that may be used according to the invention are, for example, the triethanolamine, monoethanolamine, diethanolamine, hexadecylamine, N,N,N′,N′tetrakis(2-hydroxypropyl)ethylenediamine and tris(hydroxymethyl)aminomethane salts.

As examples of 2-thioacetamide compounds of formula (I) that may be used in the invention, mention may be made of the following compounds:

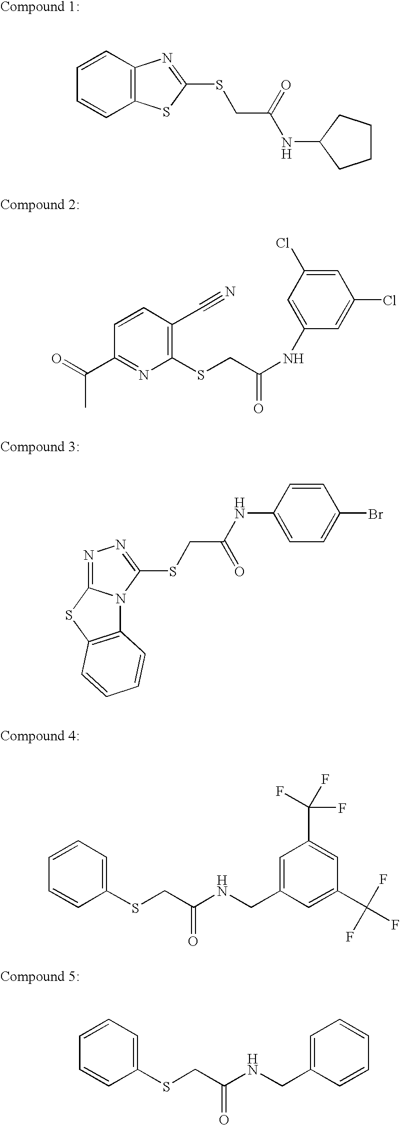

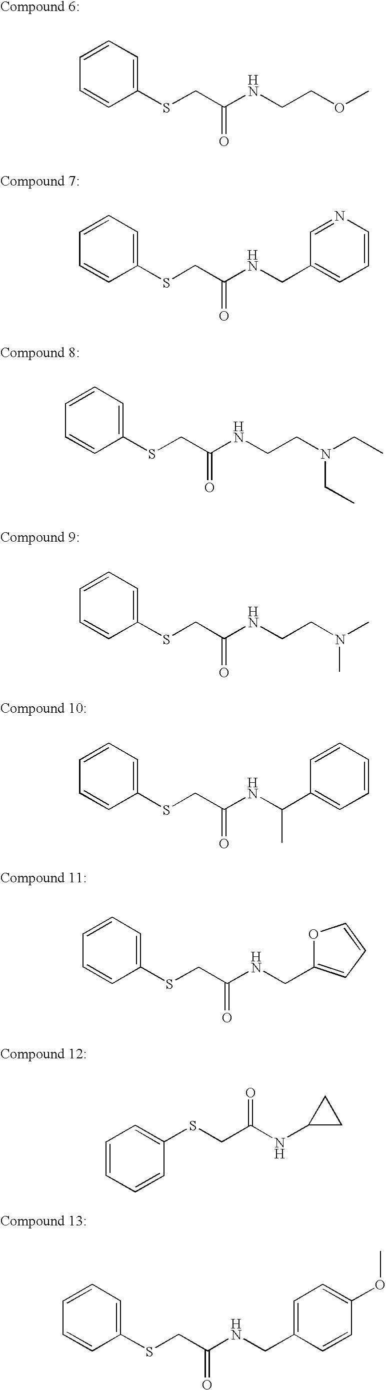

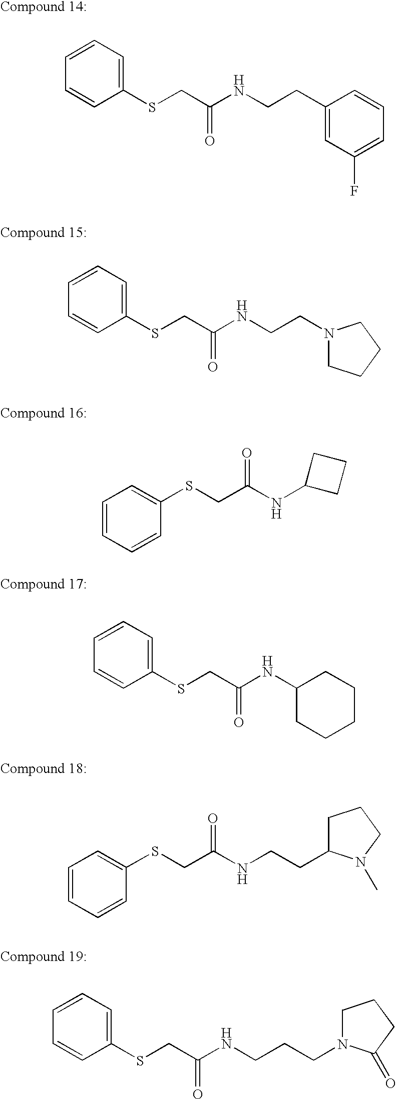

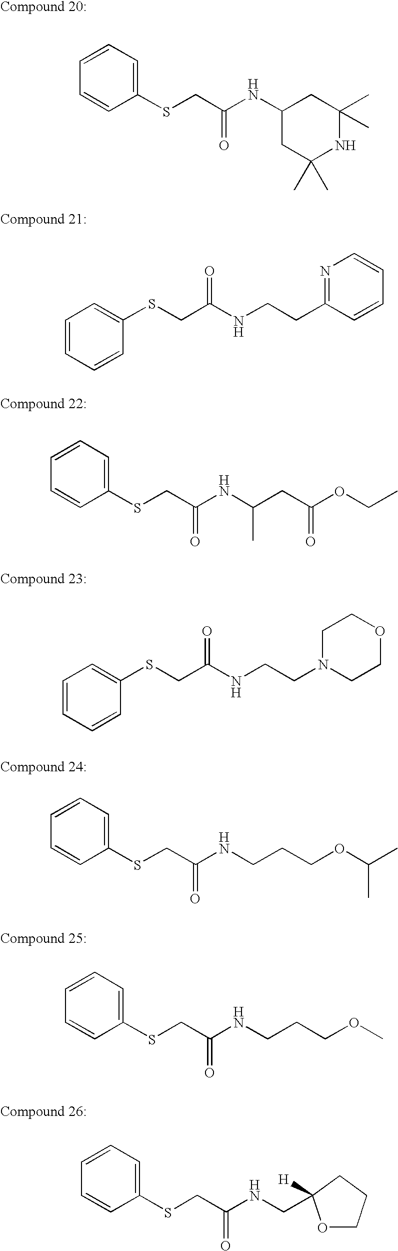

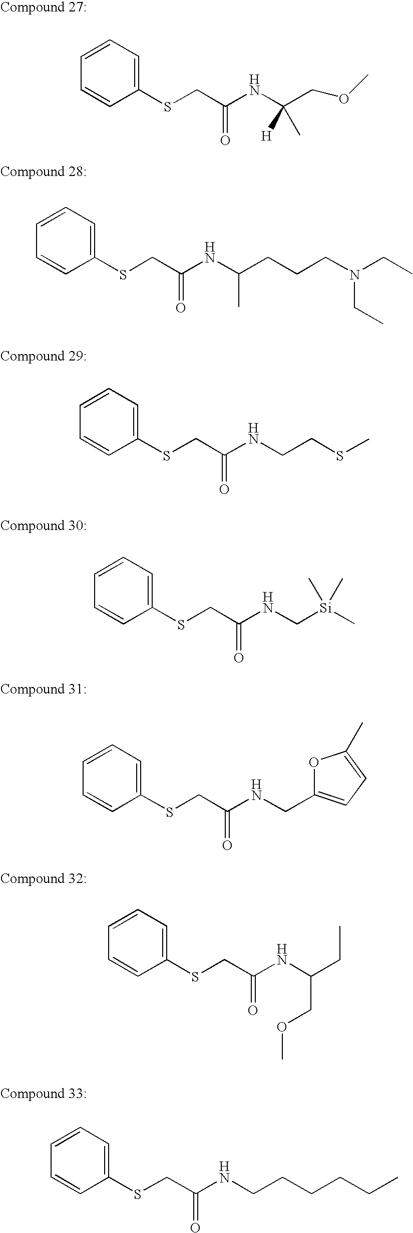

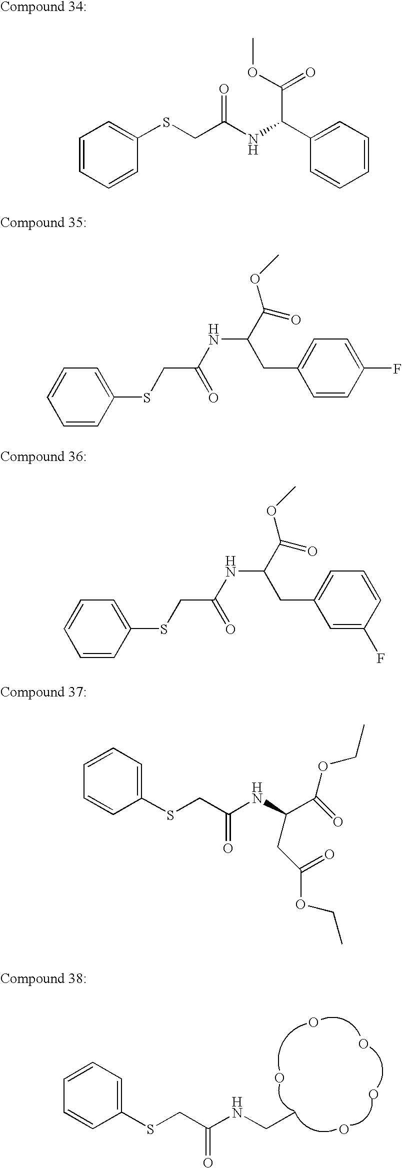

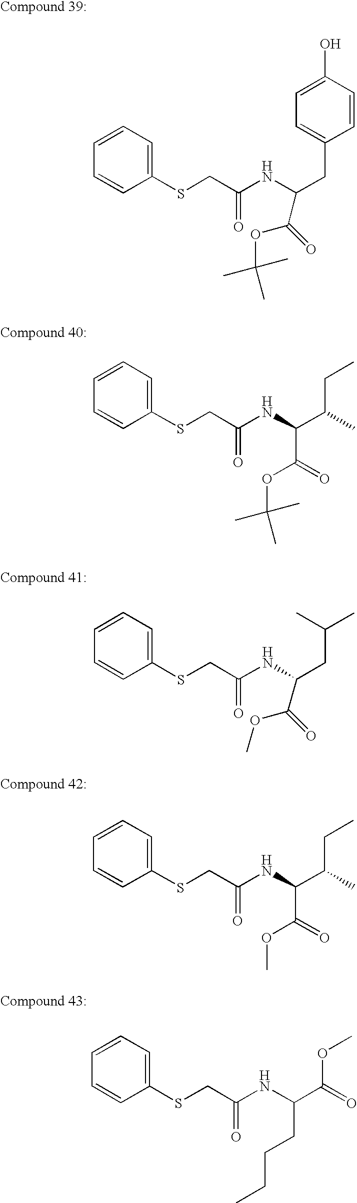

The compounds of formula (I), in salified or non-salified form, may be manufactured in a conventional manner by successive condensation of two nucleophilic molecules with the chloroacetyl chloride. For example, the amide function is made by condensing an amine with the chloroacetyl chloride in basic medium. The product obtained is itself condensed with a thiolate.

It is believed that no prior art document describes or suggests that the 2-thioacetamide compounds of formula (I) or the salts thereof have the property of inducing and/or stimulating the growth of human keratin fibers, and in particular human hair and eyelashes, and/or of reducing their loss, or that these compounds can be used topically to increase the density of human keratin fibers and more especially the hair and eyelashes.

The effective amount of a compound of formula (I) or a salt thereof corresponds to the amount required to obtain the desired result (i.e., to increase the density of keratin fibers and especially the hair and the eyelashes or to promote their growth). One skilled in the art is thus capable of evaluating this effective amount, which depends on the nature of the compound used, the person on whom it is applied and the time of this application.

In the text hereinbelow, and unless otherwise mentioned, the amounts of the various ingredients in the composition are given as weight percentages relative to the total weight of the composition.

To give an order of magnitude, according to the invention, the compound of formula (I) or a salt thereof, or a mixture of compounds of formula (I) and/or a salt thereof, may be used in an amount representing from 10⁻³% to 5% of the total weight of the composition, preferably in an amount representing from 10⁻²% to 2% of the total weight of the composition, for example from 0.5 to 2%.

The composition of the invention may be for cosmetic or pharmaceutical use. The composition of the invention is preferably for cosmetic use. In addition, the composition must contain a non-toxic, physiologically acceptable medium that can be applied to human skin, including the scalp and the eyelids and to keratin fibers. For the purposes of the invention, the term “cosmetic” means a composition of pleasant appearance, odour and feel.

The compound of formula (I), which may or may not be salified, may be used in a composition that should be ingested, injected or applied to the skin or to keratin fibers (to any area of skin or fibers to be treated).

According to the invention, the compound of formula (I) or a mixture of compounds of formula (I) may be used orally in an amount of from 0.1 to 300 mg per day, for example from 5 to 10 mg/day.

A preferred composition of the invention is a composition for cosmetic use and in particular for topical application to the skin and keratin fibers, and more especially to the scalp, the hair and the eyelashes.

This composition may be in any known presentation form that is suitable for the mode of use.

For topical application to the skin or keratin fibers, the composition may be in the form of an aqueous, alcoholic or aqueous-alcoholic solution or suspension, or an oily suspension or solution, an emulsion or dispersion of more or less fluid consistency and especially of liquid or semi-liquid consistency, obtained by dispersion of a fatty phase in an aqueous phase (O/W) or conversely (W/O), a solid (O/W) or (W/O) emulsion or dispersion, a more or less fluid or solid aqueous, aqueous-alcoholic or oily gel, a free or compacted powder to be used in unmodified form or to be incorporated into a physiologically acceptable medium, or alternatively microcapsules, microparticles or vesicular dispersions of ionic and/or nonionic type.

A composition in the form of a foam or alternatively in the form of a spray or aerosol, then comprising a pressurized propellant, may also be envisaged.

It may also be in the form a lotion, serum, milk, O/W or W/O cream, gel, unguent, ointment, powder, balm, patch, impregnated pad, cake or foam.

In particular, the composition for application to the scalp or the hair may be in the form of a haircare lotion, for example for daily or twice-weekly application, a shampoo or a hair conditioner, in particular for twice-weekly or weekly application, a liquid or solid scalp cleansing soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for topical application to the eyelashes and body hairs, the composition to which the invention applies may be in the form of a pigmented or unpigmented mascara, to be applied with a brush to the eyelashes or alternatively to beard or moustache hair.

For a composition for use by injection, the composition may be in the form of an aqueous lotion or an oily suspension. For oral use, the composition may be in the form of capsules, granules, drinkable syrups or tablets.

According to one particular embodiment, the composition according to the invention is in the form of a hair cream or hair lotion, a shampoo, a conditioner for the hair or a mascara for the hair or for the eyelashes.

The amounts of the various constituents of the physiological medium of the composition according to the invention are those generally used in the fields under consideration. In addition, these compositions are prepared according to the usual methods.

When the composition is an emulsion, the proportion of the fatty phase may range from 2% to 80% by weight and preferably from 5% to 50% by weight relative to the total weight of the composition. The aqueous phase is adjusted as a finction of the content of fatty phase and of compound(s) (I) and also of that of the optional additional ingredients, to obtain 100% by weight. In practice, the aqueous phase represents from 5% to 99.9% by weight.

The fatty phase may contain fatty or oily compounds that are liquid at room temperature (25° C.) and atmospheric pressure (760 mm Hg), which are generally known as oils. These oils may be mutually compatible or incompatible and may form a macroscopically homogeneous liquid fatty phase or a two-phase or three-phase system.

In addition to the oils, the fatty phase may contain waxes, gums, lipophilic polymers or “pasty” or viscous products containing solid parts and liquid parts.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or alternatively acetone or ether.

The emulsifiers and co-emulsifiers used to obtain a composition in emulsion form are those generally used in cosmetics and pharmaceuticals. Their nature also depends on the sense of the emulsion. In practice, the emulsifier and, where appropriate, the co-emulsifier are present in the composition in a proportion ranging from 0.1% to 30% by weight, preferably from 0.5% to 20% by weight and better still from 1% to 8% by weight. The emulsion may also contain lipid vesicles and especially liposomes.

When the composition is in the form of an oily solution or gel, the fatty phase may represent more than 90% of the total weight of the composition.

Advantageously, for a hair application, the compositions of the invention is an aqueous, alcoholic or aqueous-alcoholic solution or suspension and better still a water/ethanol solution or suspension. The alcoholic fraction may represent from 5% to 99.9% and better still from 8% to 80%.

For a mascara application, the compositions of the invention is especially in the form of a wax-in-water or wax-in-oil dispersion, a gelled oil or an aqueous gel, which may be pigmented or unpigmented.

The compositions of the invention may also comprise other additional ingredients usually used in the fields under consideration, chosen from solvents, aqueous-phase or oily-phase solvents, thickeners or gelling agents, dyes that are soluble in the medium of the composition, solid particles such as fillers or pigments, antioxidants, preserving agents, fragrances, electrolytes, neutralizers, film-forming polymers, UV blockers, for instance sunscreens, cosmetic and pharmaceutical active agents with a beneficial effect on the skin and/or keratin fibers, other than the compounds of formula (I), and mixtures thereof. These additives may be present in the composition in the amounts generally used in cosmetics and dermatology, and especially in a proportion of from 0.01% to 50% and better still from 0.1% to 20%, for example from 0.1% to 10%, relative to the total weight of the composition.

Depending on their nature, these additives may be introduced into the fatty phase, into the aqueous phase and/or into the lipid vesicles and especially liposomes.

Needless to say, one skilled in the art will take care to select the optional additional additives and/or the amount thereof such that the advantageous properties of the composition according to the invention, i.e., the inhibition of 15-PGDH and in particular the increase in the density of keratin fibers, are not, or are not substantially, adversely affected by the envisaged addition.

As solvents that may be used in the invention, mention may be made of C₂ to C₈ lower alcohols, for instance ethanol, isopropanol, propylene glycol and certain light cosmetic oils, for instance C₆ to C₁₆ alkanes.

As oils that may be used in the invention, mention may be made of oils of mineral origin (liquid petroleum jelly or hydrogenated isoparaffin), oils of plant origin (liquid fraction of shea butter, sunflower oil, apricot oil, fatty alcohol or fatty acid), oils of animal origin (perhydrosqualene), synthetic oils (fatty acid ester, purcellin oil), silicone oils (linear or cyclic polydimethylsiloxane, phenyl trimethicone) and fluoro oils (perfluoropolyethers). Waxes that may be mentioned include silicone waxes, beeswax, rice wax, candelilla wax, carnauba wax, paraffin wax and polyethylene wax.

As emulsifiers that may be used in the invention, examples that may be mentioned include glyceryl stearate, glyceryl laurate, sorbitol stearate, sorbitol oleate, alkyl dimethicone copolyols (with alkyl≧8) and mixtures thereof for a W/O emulsion. Polyethylene glycol monostearate or monolaurate, polyoxyethylenated sorbitol stearate or oleate, and dimethicone copolyols, and mixtures thereof, may also be used for an O/W emulsion.

As hydrophilic gelling agents that may be used in the invention, mention may be made of carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents that may be used, mention may be made of modified clays, for instance Bentones, metal salts of fatty acids, for instance aluminium stearates, hydrophobic-treated silica and ethylcellulose, and mixtures thereof.

As cosmetic or pharmaceutical active agent other than the compound of formula (I), which may be used in the invention, mention may be made of hydrophilic active agents chosen from proteins or protein hydrolysates, amino acids, polyols, urea, allantoin, sugars and sugar derivatives, water-soluble vitamins, plant extracts (those from Iridacea plants or from soybean) and hydroxy acids such as fruit acids or salicylic acid; or lipophilic active agents such as retinol (vitamin A) and its derivatives, especially an ester (retinyl palmitate), tocopherol (vitamin E) and its derivatives, especially an ester (tocopheryl acetate), essential fatty acids, ceramides, essential oils, salicylic acid derivatives, for instance 5-n-octanoylsalicylic acid, hydroxy acid esters, and phospholipids, for instance lecithin, and mixtures thereof.

According to one particular embodiment of the invention, the compound of formula (I) may be combined with at least one additional active compound that promotes the regrowth and/or limits the loss of keratin fibers (hair or eyelashes). These additional compounds are chosen especially from the lipoxygenase inhibitors as described in EP-0,648,488, the bradykinin inhibitors described especially in EP-0,845,700, prostaglandins and derivatives thereof, especially those described in WO 98/33497, WO 95/11003, JP 97-100 091 and JP 96-134 242, prostaglandin receptor agonists or antagonists, the non-prostanoic prostaglandin analogues as described in EP-1,175,891, EP-1,175,890, WO 01/74307, WO 01/74313, WO 01/74314, WO 01/74315 or WO 01/72268, and mixtures thereof.

As other additional active compounds that promote the growth of keratin fibers and/or reduce their loss, which may be present in the composition according to the invention, mention may be made of vasodilators, antiandrogens, cyclosporins and analogues thereof, antimicrobial and antifungal agents, anti-inflammatory agents, and retinoids, alone or in a mixture.

The vasodilators that may be used are especially potassium-channel agonists, including minoxidil, and also the compounds described in patents U.S. Pat. Nos. 3,382,247, 5,756,092, 5,772,990, 5,760,043, 5,466,694, 5,438,058 and 4,973,474, cromakalim, nicorandil and diaxozide, alone or in combination.

The antiandrogens that may be used especially include steroidal and non-steroidal 5α-reductase inhibitors, for instance finasteride and the compounds described in U.S. Pat. No. 5,516,779, cyprosterone acetate, azelaic acid and the salts and derivatives thereof, and the compounds described in U.S. Pat. No. 5,480,913, flutamide, oxendolone, spironolactone, diethylstilbestrol and the compounds described in U.S. Pat. Nos. 5,411,981, 5,565,467 and 4,910,226.

The antimicrobial or antifungal compounds may be chosen from selenium derivatives, octopirox, triclocarban, triclosan, zinc pyrithione, itraconazole, asiatic acid, hinokitiol, mipirocine, tetracyclines, especially erythromycin and the compounds described in EP 0 680 745, clinycin hydrochloride, benzoyl peroxide or benzyl peroxide, minocycline and compounds belonging to the imidazole class, such as econazole, ketoconazole or miconazole or salts thereof, nicotinic acid esters, especially including tocopheryl nicotinate, benzyl nicotinate and C₁-C₆ alkyl nicotinates, for instance methyl nicotinate or hexyl nicotinate.

The anti-inflammatory agents may be chosen from steroidal anti-inflammatory agents, for instance glucocorticoids, corticosteroids (for example: hydrocortisone) and non-steroidal anti-inflammatory agents, for instance glycyrrhetinic acid and α-bisabolol, benzydamine, salicylic acid and the compounds described in EP-0,770,399, WO 94/06434 and FR-2,268,523.

The retinoids may be chosen from isotretinoin, acitretin and tazarotene.

As other active compounds for promoting the growth and/or limiting the loss of hair that may be used in combination with the compound of formula (I), which may or may not be salified, mention may be made of aminexil, 6-0-[(9Z,12Z)octadeca-9,12-dienoyl]hexapyranose, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharide or acylhexosaccharide acids, substituted aryl ethylenes, N-acylamino acids, flavonoids, ascomycin derivatives and analogues, histamine antagonists, saponins, proteoglycanase inhibitors, oestrogen agonists and antagonists, pseudoterines, cytokines, growth factor promoters, IL-1 or IL-6 inhibitors, IL-10 promoters, TNF inhibitors, benzophenones, hydantoin, retinoic acid; vitamins, for instance vitamin D, vitamin B12 analogues and pantothenol; triterpenes, for instance ursolic acid and the compounds described in U.S. Pat. Nos. 5,529,769, 5,468,888 and 5,631,282; antipruriginous agents, for instance thenaldine, trimeprazine or cyproheptadine; antiparasitic agents, in particular metronidazole, crotamiton or pyrethroids; calcium antagonists, for instance cinnarizine, diltiazem, nimodipine, verapamil, alverine and nifedipine; hormones such as oestriol or its analogues, thyroxine and its salts, and progesterone; FP receptor (type-F prostaglandin receptor) antagonists such as latanoprost, bimatoprost, travoprost or unoprostone; mixtures thereof.

Advantageously, the compositions according to the invention comprise at least one 15-PGDH inhibitor as defined above and at least one prostaglandin or prostaglandin derivative, for instance the prostaglandins of series 2 especially including PGF2-α and PGE2 in salt or ester form (for example the isopropyl esters), derivatives thereof, for instance 16,16-dimethyl PGE2,17-phenyl PGE2,16,16-dimethyl PGF2-α, 17-phenyl PGF2-α, prostaglandins of series 1, for instance 11-deoxyprostaglandin E1, 1-deoxyprostaglandin E1 in salt or ester form, analogues thereof, especially latanoprost, fluprostenol, bimatoprost, cloprostenol, viprostol, butaprost, misoprostol, unoprostone, and the salts or esters thereof.

The compositions advantageously contain at least one non-prostanoic EP2 and/or EP4 receptor agonist as described especially in EP-1,175,892.

It may also be envisaged for the composition comprising at least the compound of formula (I), salified or non-salified, to be in liposomal form, as described especially in document WO 94/22468. Thus, the compound encapsulated in the liposomes may be delivered selectively to the hair follicle.

The compositions according to the invention may be applied to the alopecic areas of the scalp and the hair of an individual, and optionally left in contact for several hours and optionally rinsed off.

The compositions containing an effective amount of a compound of formula (I), salified or non-salified, may, for example, be applied in the evening, kept in contact throughout the night and optionally shampooed out in the morning. These applications may be repeated daily for one or more months according to the individual.

Thus, the present invention also features a cosmetic process (regime or regimen) for treating human keratin fibers and/or the skin from which the said fibers emerge, including the scalp and the eyelids, comprising applying to the keratin fibers and/or the skin from which the said fibers emerge a cosmetic composition comprising at least one derivative of compound (I) or a salt thereof, leaving the said composition in contact with the keratin fibers and/or the skin from which the said fibers emerge, and optionally rinsing the fibers and/or the skin.

This treatment process does indeed have the characteristics of a cosmetic process since it makes it possible to enhance the appearance of human keratin fibers and to give them greater vigour and an improved look. In addition, it may be used daily for several months, without medical prescription.

More especially, the present invention features a cosmetic care process for human hair and/or the scalp, to improve their condition and/or look, comprising applying to the hair and/or the scalp a cosmetic composition comprising an effective amount of at least one compound of the formula (I) or a salt thereof, leaving the said composition in contact with the hair and/or the scalp, and optionally rinsing the hair and/or the scalp.

Advantageously, in the process according to the invention, between 5 and 500 μl of a solution or composition as defined above, comprising from 0.001% to 5% of 15-PGDH inhibitor, is applied to the areas of the scalp to be treated.

This invention also features a cosmetic care and/or makeup process for human eyelashes, to improve their condition and/or their appearance, comprising applying to the eyelashes and/or the eyelids a mascara composition comprising at least one compound of formula (I) or a salt thereof, and leaving the said composition in contact with the eyelashes and/or the eyelids. This mascara composition may be applied alone or as a basecoat for a standard pigmented mascara, and may be removed like a standard pigmented mascara.

The present invention also features a care or makeup composition for keratin fibers, comprising, in a physiologically acceptable medium, in particular a cosmetic medium, at least one derivative of formula (I) or a salt thereof and at least one additional active agent that promotes the regrowth of human keratin fibers and/or that limits their loss, chosen from aminexil, FP receptor agonists and vasodilators, and more especially chosen from aminexil, minoxidil, latanoprost and travoprost.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Reaction Scheme for the Synthesis of Compound 1

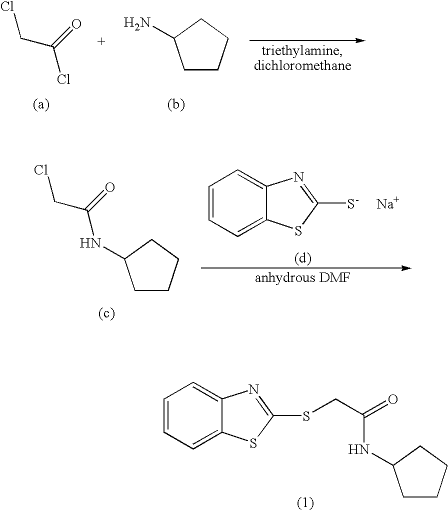

Procedure

Cyclopentylamine (compound b) (11.65 mL, 0.117 mol) is diluted in 100 mL of dichloromethane in a 250 mL three-necked flask equipped with a condenser, a thermometer, an addition funnel and a magnetic stirrer. Triethylamine (18.15 mL, 0.129 mol) is added and the medium is then cooled to 10° C. The chloroacetyl chloride (compound a) (9.35 mL, 0.117 mol), diluted beforehand in 25 mL of dichloromethane, is then added dropwise while keeping the temperature between 10° C. and 15° C. After this addition, the reaction medium is stirred at room temperature for 1 hour. The mixture is then washed with water (2×100 mL), with 1N hydrochloric acid solution (2×50 mL) and then with water (2×100 mL) and finally with saturated sodium chloride solution. The organic phase is dried over sodium sulfate, filtered and then concentrated to the maximum. 15.5 g of a brown solid (compound c) are obtained in a yield of 82%.

The brown solid (8.51 g, 53 mmol) obtained above is dissolved in 100 mL of anhydrous dimethylformamide (DMF) in a 250 mL three-necked flask equipped with a condenser, a thermometer, an argon inlet and a magnetic stirrer, followed by addition of the sodium salt of 2-mercaptobenzothiazole (compound d) (10 g, 53 mmol). The reaction medium is heated at 60° C. for 5 hours and then concentrated to the maximum. The mixture is then diluted with 100 mL of dichloromethane, and then washed with water (3×100 mL), with 2N hydrochloric acid solution (1×50 mL) and then with water (2×100 mL) and finally with saturated sodium chloride solution. The organic phase is dried over sodium sulfate, filtered and then concentrated to the maximum to give a brown solid. This solid is taken up in 200 mL of diethyl ether and stirred at room temperature for 2 hours. The solid obtained is filtered off on a sinter funnel and then dried under vacuum in the presence of phosphorus pentoxide. Product 1 is obtained in the form of a beige-coloured solid (11.5 g) in a yield of 74%.

Mass spectrometry: The quasi-molecular ions (MH)⁺, (MNa)⁺ and (M-H)⁻ of the expected molecule, C₁₄H₁₆N₂OS₂, are mainly detected.

Nuclear Magnetic Resonance: The spectra obtained are in accordance with the proposed structure; resolution of certain signals due to the syn and anti form of the amide. The resolution disappears on heating. ¹H NMR (CDCl₃) δ: 1.38 (m, 2H); 1.54 (m, 4H); 1.88 (m, 2H); 3.89 (s, 2H); 4.17 (m, 1H); 7.33 (m, 1H); 7.44 (m, 1H); 7.53 (se, 1H); 7.76 (d, 1H); 7.82 (d, 1H).

### EXAMPLES 2 AND 3

Compounds 2 and 3 may be synthesized via a synthetic method analogous to that for compound 1.

### EXAMPLES 4 TO 43

### Reaction Scheme for the Synthesis of Compounds 4 to 43

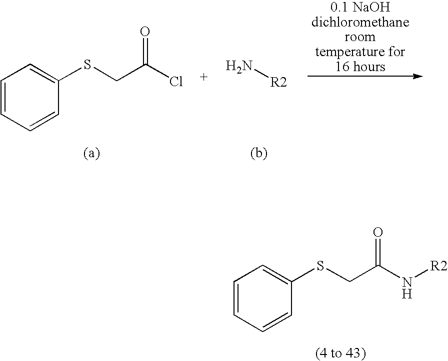

A solution of 0.022 mmol of amine in 500 μL of dichloromethane is added to 0.02 mmol of compound (a) dissolved in 500 μL of dichloromethane. 1 ml of 0.1N sodium hydroxide solution is added to the reaction medium and stirring is continued overnight at room temperature. The reaction medium is dried by passing it through a Chem Elut® cartridge (Varian) and rinsed with 5 mL of dichloromethane. The solvent is evaporated off. The amide is obtained in the form of a solid (40-100% yield).

### EXAMPLE 44

### Demonstration of the 15-PGDH-Specific Inhibitory Properties of the Compounds of Formula (1)

1) Test on 15-PGDH

The enzyme 15-PGDH is obtained as described in patent application FR 02/05067 filed in the name of L'Oréal, as a suspension in a medium adjusted to a concentration of 0.3 mg/mL and then blocked at −80° C. For the purposes of the test, this suspension is thawed and stored in ice.

In parallel, a 100 mM, pH 7.4 Tris buffer containing 0.1 mM of dithiothreitol (D5545, Sigma-Aldrich, L'isle D'Abeau Chesne, BP 701, 38297, Saint Quentin Fallavier), 1.5 mM of β-NAD (N6522, Sigma-Aldrich, L'isle D'Abeau Chesne, BP 701, 38297, Saint Quentin Fallavier), and 50 μM of prostaglandin E₂ (P4172, Sigma-Aldrich, L'isle D'Abeau Chesne, BP 701, 38297, Saint Quentin Fallavier) is prepared.

0.965 ml of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.035 mL of enzymatic suspension at 37° C. is introduced into the cuvette concomitantly with the recording (corresponding to an increase in the optical density at 340 nm). The maximum reaction rate is recorded.

The test values (containing the compounds (I)) are compared with the control value (without compound (I)); the results indicated represent the concentration at which the compound of formula (I) reduces the anzymatic activity of 15-PGDH by 50%, namely IC_{50dh}.

2) Test on PGF Synthase

The enzyme PGFS is obtained as described in document FR-A-02/05067, at a concentration of 0.5 mg/mL, as a suspension in a suitable medium, and blocked at −80° C. For the purposes of the test, this suspension is thawed and stored in ice.

In parallel, a 100 mM, pH 6.5 Tris buffer containing 20 μM of 9,10-phenanthrenequinone* (P2896, Sigma-Aldrich, L'isle D'Abeau Chesne, BP 701, 38297, Saint Quentin Fallavier) and 100 μM of β-NADPH (N1630, Sigma-Aldrich, L'isle D'Abeau Chesne, BP 701, 38297, Saint Quentin Fallavier) is prepared in a brown flask (protected from light).

*A stock solution with a titre of 1 mM is prepared in absolute ethanol and brought to 40° C.; the flask is placed in an ultrasound cuvette to facilitate the dissolution of the product.

0.950 mL of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.05 mL of enzymatic suspension at 37° C. is introduced into the cuvette concomitantly with the recording (corresponding to a reduction in the optical density at 340 nm). The maximum reaction rate is recorded.

The test values (containing compound (I)) are compared with the control value (without compound (I)); the results indicated represent the concentration at which the compound of formula (I) reduces the enzymatic activity of PGFS by 50%, namely IC_{50fs}.

| | | Inhibition of 15-PGDH | | PGFS |
|---|---|---|---|---|
| | | IC₅₀ | % inhibition at | IC₅₀ |
| Compound | Structure | (μm) | 50 μm | (μm) |
| 1 | 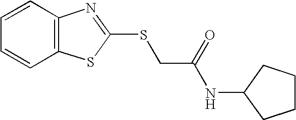 | 5 | 89 | ≧50 |
| 2 | 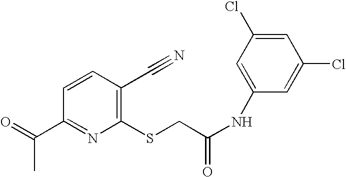 | | 30 | |
| 3 | 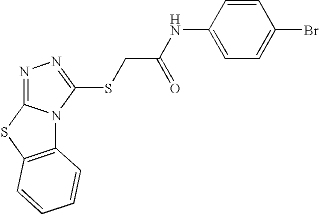 | | 26 | |

From this table, it is seen that compound 1 is indeed a 15-P GDH inhibitor. Furthermore, it inhibits 15-PGDH more efficiently and more selectively than PGFS. Thus, the IC_{50fs}/IC_{50dh} ratio is greater than 10.

The compositions below are obtained via the usual techniques commonly used in cosmetics or pharmaceuticals.

In each of the formulation examples, a fluid that may be used by application once or twice a day to the scalp to reduce hair loss and/or promote hair growth is obtained.

### EXAMPLE 45

### Hair Lotion

| | | |
|---|---|---|
| Compound of Example 1 | 1.00 | g |
| Propylene glycol | 30.00 | g |
| Ethyl alcohol | 40.00 | g |
| Water | qs 100.00 | g |

This lotion is applied to the scalp, once or twice a day, at a rate of 1 mL per application.

### EXAMPLE 46

### Hair Lotion

| | | |
|---|---|---|
| Compound 1 | 1.00 | g |
| Propylene glycol | 30.00 | g |
| Ethyl alcohol | 40.00 | g |
| Water | qs 100.00 | g |

This lotion is applied to the scalp, once or twice a day, at a rate of 1 mL per application, massaging the scalp gently to help the active agent to penetrate. The head of hair is then dried in the open air. This lotion makes it possible to reduce hair loss and to promote regrowth of the hair.

### EXAMPLE 47

### Wax/Water Mascara

| | |
|---|---|
| Beeswax | 6.00% |
| Paraffin wax | 13.00% |
| Hydrogenated jojoba oil | 2% |
| Water-soluble film-forming polymer | 3% |
| Triethanolamine stearate | 8% |
| Compound 1 | 1% |
| Black pigment | 5% |
| Preserving agent | qs |
| Water | qs |
| | 100% |

This mascara is applied to the eyelashes like a standard mascara with a mascara brush.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A regime or regimen for inducing and/or stimulating the growth of human keratin fibers and/or reducing the loss and/or increasing the density thereof, comprising administering to an individual in need of such treatment, a thus-effective amount of at least one 2-thioacetamide compound of formula (I), or salt thereof: 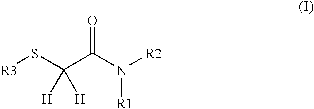
in which:
a) R₁ and R₂ independently represent:
1) a hydrogen atom,
2) a C₁-C₂₀ alkyl radical, optionally substituted with at least one substituent A₁,
3) a hydrocarbon-based ring C₁, optionally fused to a ring C₂ optionally containing at least one hetero atom to form a heterocycle Hy₁, these rings C₁ and C₂ optionally bearing at least one carbonyl or thiocarbonyl function and being substituted with at least one substituent A₃,
4) a heterocycle Hy₂, optionally fused to a ring C₂ optionally containing at least one hetero atom to form a heterocycle Hy₃, these rings Hy₂ and C₂ optionally bearing a carbonyl or thiocarbonyl function and being substituted with at least one substituent A₃, or
5) a group C(═NR)R′, C(═NR)NR′R″, COR, CSR, COOR, CONRR′, SO₂R or SO₂NRR′;
b) R₃ represents:
1) a C₁-C₂₀ alkyl radical, optionally substituted with at least one substituent A₂,
2) a hydrocarbon-based ring C₃, optionally fused to a ring C₄ optionally containing at least one hetero atom to form a heterocycle Hy₃, these rings C₃ and C₄ optionally bearing at least one carbonyl or thiocarbonyl function and being substituted with at least one substituent A₃,
3) a heterocycle Hy₄ representing a pyrrole, furan, thiophene or pyrazole ring, optionally fused to a ring C₅ representing a phenyl, pyridine or pyrimidine ring, these two rings Hy₄ and C₅ optionally being substituted with at least one substituent A₃,
4) a pyridine ring optionally fused to a ring C₂ optionally containing at least one hetero atom to form a heterocycle Hy₁, these pyridine and C₂ rings optionally bearing at least one carbonyl or thiocarbonyl function and being substituted with at least one substituent A₄,
5) a heterocycle Hy₅ other than Hy₄ and the pyridine ring, optionally fused to a ring C₂ optionally containing at least one hetero atom to form a heterocycle Hy₁, these rings Hy₅ and C₂ optionally bearing at least one carbonyl or thiocarbonyl function and being substituted with at least one substituent A₃, or
6) a group C(═NR)R′, C(═NR)NR′R″, COR, CSR, COOR or CONRR′;
c) A₁ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a hydrocarbon-based ring C₆, optionally fused to a ring C₇ optionally containing at least one hetero atom to form a heterocycle Hy₆, these rings C₆ and C₇ optionally bearing at least one carbonyl or thiocarbonyl function and being substituted with at least one substituent A₅,
4) a heterocycle Hy₇ optionally fused to a ring C₇ optionally containing at least one hetero atom to form a heterocycle Hy₆, these rings Hy₇ and C₇ optionally bearing at least one carbonyl or thiocarbonyl function and being substituted with at least one substituent A₅;
d) A₂ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″, or
3) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
e) A₃ represents:
1) A₂, or
2) a C₁-C₂₀ alkyl group optionally substituted with at least one substituent A₅;
f) A₄ represents:
1) a halogen,
2) a group CF₃, CN, OR, SR, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a C₁-C₂₀ alkyl group optionally substituted with at least one substituent A₅,
4) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
g) A₅ represents:
1) a halogen,
2) a group CF₃, CN, OR₅, SR₅, NR₅R₅′, NR₅C(═NR₅′)NR₅″R₅′″, COR₅, CSR₅, COOR₅, CONR₅R₅′, CSNR₅R₅′, NR₅CSR₅′, NR₅CSNR₅′R₅″, NR₅COR₅′, NR₅CONR₅′R₅″, SO₂NR₅R₅′, NR₅SO₂R₅′, SO₂R₅ or SiR₅R₅′R₅″, R₅, R₅′, R₅″ being a hydrogen atom or a C₁-C₂₀ alkyl group,
3) a C₁-C₂₀ alkyl group, or
4) a ring C₁₀ optionally fused to another ring C₁₁, these rings optionally containing at least one hetero atom to form a heterocycle Hy₉ and/or bearing at least one carbonyl or thiocarbonyl function;
h) R, R′, R″ and R′″, which may be identical or different, represent:
1) a hydrogen atom,
2) a C₁-C₂₀ alkyl group optionally substituted with at least one substituent A₅, or
3) a ring C₁₂ optionally fused to another ring C₁₃, these rings optionally containing at least one hetero atom to form a heterocycle Hy₁₀ and being optionally substituted with at least one substituent A₅;
i) Hy₁ to Hy₁₀ independently represent a heterocycle optionally containing from 1 to 4 hetero atoms selected from the group consisting of N, O and S.

2. A regime or regimen for inhibiting 1.5-hydroxyprostaglandin dehydrogenase, comprising administering to an individual in need of such treatment, a thus-effective amount of at least one 2-thioacetamide compound of formula (I), or salt thereof: 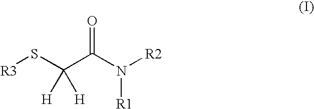
in which:
a) R₁ and R₂ independently represent:
1) a hydrogen atom,
2) a C₁-C₂₀ alkyl radical, optionally substituted with at least one substituent A₁,
3) a hydrocarbon-based ring C₁, optionally fused to a ring C₂ optionally containing at least one hetero atom to form a heterocycle Hy₁, these rings C₁ and C₂ optionally bearing at least one carbonyl or thiocarbonyl function and being substituted with at least one substituent A₃,
4) a heterocycle Hy₂, optionally fused to a ring C₂ optionally containing at least one hetero atom to form a heterocycle Hy₃, these rings Hy₂ and C₂ optionally bearing a carbonyl or thiocarbonyl function and being substituted with at least one substituent A₃, or
5) a group C(═NR)R′, C(═NR)NR′R″, COR, CSR, COOR, CONRR′, SO₂R or SO₂NRR′;
b) R₃ represents:
1) a C₁-C₂₀ alkyl radical, optionally substituted with at least one substituent A₂,
2) a hydrocarbon-based ring C₃, optionally fused to a ring C₄ optionally containing at least one hetero atom to form a heterocycle Hy₃, these rings C₃ and C₄ optionally bearing at least one carbonyl or thiocarbonyl function and being substituted with at least one substituent A₃,
3) a heterocycle Hy₄ representing a pyrrole, furan, thiophene or pyrazole ring, optionally fused to a ring C₅ representing a phenyl, pyridine or pyrimidine ring, these two rings Hy₄ and C₅ optionally being substituted with at least one substituent A₃,
4) a pyridine ring optionally fused to a ring C₂ optionally containing at least one hetero atom to form a heterocycle Hy₁, these pyridine and C₂ rings optionally bearing at least one carbonyl or thiocarbonyl function and being substituted with at least one substituent A₄,
5) a heterocycle Hy₅ other than Hy₄ and the pyridine ring, optionally fused to a ring C₂ optionally containing at least one hetero atom to form a heterocycle Hy₁, these rings Hy₅ and C₂ possibly bearing at least one carbonyl or thiocarbonyl function and being substituted with at least one substituent A₃, or
6) a group C(═NR)R′, C(═NR)NR′R″, COR, CSR, COOR or CONRR′;
c) A₁ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a hydrocarbon-based ring C₆, optionally fused to a ring C₇ optionally containing at least one hetero atom to form a heterocycle Hy₆, these rings C₆ and C₇ optionally bearing at least one carbonyl or thiocarbonyl function and being substituted with at least one substituent A₅,
4) a heterocycle Hy₇ optionally fused to a ring C₇ optionally containing at least one hetero atom to form a heterocycle Hy₆, these rings Hy₇ and C₇ optionally bearing at least one carbonyl or thiocarbonyl function and being substituted with at least one substituent A₅;
d) A₂ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″, or
3) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
e) A₃ represents:
1) A₂, or
2) a C₁-C₂₀ alkyl group optionally substituted with at least one substituent A₅;
f) A₄ represents:
1) a halogen,
2) a group CF₃, CN, OR, SR, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a C₁-C₂₀ alkyl group optionally substituted with at least one substituent A₅,
4) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
g) A₅ represents:
1) a halogen,
2) a group CF₃, CN, OR₅, SR₅, NR₅R₅′, NR₅C(═NR₅′)NR₅″R₅′″, COR₅, CSR₅, COOR₅, CONR₅R₅′, CSNR₅R₅′, NR₅CSR₅′, NR₅CSNR₅′R₅″, NR₅COR₅′, NR₅CONR₅′R₅″, SO₂NR₅R₅′, NR₅SO₂R₅′, SO₂R₅ or SiR₅R₅′R₅″, R₅, R₅′, R₅″ being a hydrogen atom or a C₁-C₂₀ alkyl group,
3) a C₁-C₂₀ alkyl group, or
4) a ring C₁₀ optionally fused to another ring C₁₁, these rings optionally containing at least one hetero atom to form a heterocycle Hy₉ and/or bearing at least one carbonyl or thiocarbonyl function;
h) R, R′, R″ and R′″, which may be identical or different, represent:
1) a hydrogen atom,
2) a C₁-C₂₀ alkyl group optionally substituted with at least one substituent A₅, or
3) a ring C₁₂ optionally fused to another ring C₁₃, these rings optionally containing at least one hetero atom to form a heterocycle Hy₁₀ and being optionally substituted with at least one substituent A₅;
i) Hy₁ to Hy₁₀ independently represent a heterocycle optionally containing from 1 to 4 hetero atoms selected from the group consisting of N, O and S.

3. The regime or regimen as defined by claim 1, said human keratin fibers comprising head hair, the eyebrows, the eyelashes, beard hair, moustache hair and/or pubic hair.

4. A regime or regimen for treating androchronogenetic alopecia and for treating alopecioa of natural origin, comprising administering to an individual in need of such treatment, a thus-effective amount of at least one 2-thioacetamide compound of formula (I), or salt thereof: 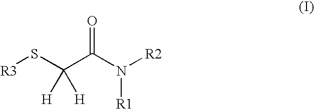
in which:
a) R₁ and R₂ independently represent:
1) a hydrogen atom,
2) a C₁-C₂₀ alkyl radical, optionally substituted with at least one substituent A₁,
3) a hydrocarbon-based ring C₁, optionally fused to a ring C₂ optionally containing at least one hetero atom to form a heterocycle Hy₁, these rings C₁ and C₂ optionally bearing at least one carbonyl or thiocarbonyl function and being substituted with at least one substituent A₃,
4) a heterocycle Hy₂, optionally fused to a ring C₂ optionally containing at least one hetero atom to form a heterocycle Hy₃, these rings Hy₂ and C₂ optionally bearing a carbonyl or thiocarbonyl function and being substituted with at least one substituent A₃, or
5) a group C(═NR)R′, C(═NR)NR′R″, COR, CSR, COOR, CONRR′, SO₂R or SO₂NRR′;
b) R₃ represents:
1) a C₁-C₂₀ alkyl radical, optionally substituted with at least one substituent A₂,
2) a hydrocarbon-based ring C₃, optionally fused to a ring C₄ optionally containing at least one hetero atom to form a heterocycle Hy₃, these rings C₃ and C₄ optionally bearing at least one carbonyl or thiocarbonyl function and being substituted with at least one substituent A₃,
3) a heterocycle Hy₄ representing a pyrrole, furan, thiophene or pyrazole ring, optionally fused to a ring C₅ representing a phenyl, pyridine or pyrimidine ring, these two rings Hy₄ and C₅ optionally being substituted with at least one substituent A₃,
4) a pyridine ring optionally fused to a ring C₂ optionally containing at least one hetero atom to form a heterocycle Hy₁, these pyridine and C₂ rings optionally bearing at least one carbonyl or thiocarbonyl function and being substituted with at least one substituent A₄,
5) a heterocycle Hy₅ other than Hy₄ and the pyridine ring, optionally fused to a ring C₂ optionally containing at least one hetero atom to form a heterocycle Hy₁, these rings Hy₅ and C₂ possibly bearing at least one carbonyl or thiocarbonyl function and being substituted with at least one substituent A₃, or
6) a group C(═NR)R′, C(═NR)NR′R″, COR, CSR, COOR or CONRR′;
c) A₁ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a hydrocarbon-based ring C₆, optionally fused to a ring C₇ optionally containing at least one hetero atom to form a heterocycle Hy₆, these rings C₆ and C₇ optionally bearing at least one carbonyl or thiocarbonyl function and being substituted with at least one substituent A₅,
4) a heterocycle Hy₇ optionally fused to a ring C₇ optionally containing at least one hetero atom to form a heterocycle Hy₆, these rings Hy₇ and C₇ optionally bearing at least one carbonyl or thiocarbonyl function and being substituted with at least one substituent A₅;
d) A₂ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″, or
3) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
e) A₃ represents:
1) A₂, or
2) a C₁-C₂₀ alkyl group optionally substituted with at least one substituent A₅;
f) A₄ represents:
1) a halogen,
2) a group CF₃, CN, OR, SR, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a C₁-C₂₀ alkyl group optionally substituted with at least one substituent A₅,
4) a ring C₈ optionally fused to a ring C₉, these rings C₈ and C₉ optionally containing at least one hetero atom to form a heterocycle Hy8 and/or at least one carbonyl or thiocarbonyl function and/or being substituted with at least one substituent A₅;
g) A₅ represents:
1) a halogen,
2) a group CF₃, CN, OR₅, SR₅, NR₅R₅′, NR₅C(═NR₅′)NR₅″R₅′″, COR₅, CSR₅, COOR₅, CONR₅R₅′, CSNR₅R₅′, NR₅CSR₅′, NR₅CSNR₅′R₅″, NR₅COR₅′, NR₅CONR₅′R₅″, SO₂NR₅R₅′, NR₅SO₂R₅′, SO₂R₅ or SiR₅R₅′R₅″, R₅, R₅′, R₅″ being a hydrogen atom or a C₁-C₂₀ alkyl group,
3) a C₁-C₂₀ alkyl group, or
4) a ring C₁₀ optionally fused to another ring C₁₁, these rings optionally containing at least one hetero atom to form a heterocycle Hy₉ and/or bearing at least one carbonyl or thiocarbonyl function;
h) R, R′, R″ and R′″, which may be identical or different, represent:
1) a hydrogen atom,
2) a C₁-C₂₀ alkyl group optionally substituted with at least one substituent A₅, or
3) a ring C₁₂ optionally fused to another ring C₁₃, these rings optionally containing at least one hetero atom to form a heterocycle Hy₁₀ and being optionally substituted with at least one substituent A₅;
i) Hy₁ to Hy₁₀ independently represent a heterocycle optionally containing from 1 to 4 hetero atoms selected from the group consisting of N, O and S.

5. A regime or regimen for the care and/or makeup of human eyelashes, for inducing and/or stimulating the growth and/or increasing the density thereof, comprising administering to an individual in need of such treatment, a thus-effective amount of at least one 2-thioacetamide compound of formula (I), or salt thereof: 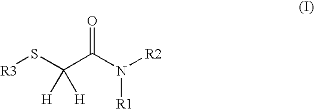
in which:
a) R₁ and R₂ independently represent:
1) a hydrogen atom,
2) a C₁-C₂₀ alkyl radical, optionally substituted with at least one substituent A₁,
3) a hydrocarbon-based ring C₁, optionally fused to a ring C₂ optionally containing at least one hetero atom to form a heterocycle Hy₁, these rings C₁ and C₂ optionally bearing at least one carbonyl or thiocarbonyl function and being substituted with at least one substituent A₃,
4) a heterocycle Hy₂, optionally fused to a ring C₂ optionally containing at least one hetero atom to form a heterocycle Hy₃, these rings Hy₂ and C₂ optionally bearing a carbonyl or thiocarbonyl function and being substituted with at least one substituent A₃, or
5) a group C(═NR)R′, C(═NR)NR′R″, COR, CSR, COOR, CONRR′, SO₂R or SO₂NRR′;
b) R₃ represents:
1) a C₁-C₂₀ alkyl radical, optionally substituted with at least one substituent A₂,
2) a hydrocarbon-based ring C₃, optionally fused to a ring C₄ optionally containing at least one hetero atom to form a heterocycle Hy₃, these rings C₃ and C₄ optionally bearing at least one carbonyl or thiocarbonyl function and being substituted with at least one substituent A₃,
3) a heterocycle Hy₄ representing a pyrrole, furan, thiophene or pyrazole ring, optionally fused to a ring C₅ representing a phenyl, pyridine or pyrimidine ring, these two rings Hy₄ and C₅ optionally being substituted with at least one substituent A₃,
4) a pyridine ring optionally fused to a ring C₂ optionally containing at least one hetero atom to form a heterocycle Hy₁, these pyridine and C₂ rings optionally bearing at least one carbonyl or thiocarbonyl function and being substituted with at least one substituent A₄,
5) a heterocycle Hy₅ other than Hy₄ and the pyridine ring, optionally fused to a ring C₂ optionally containing at least one hetero atom to form a heterocycle Hy₁, these rings Hy₅ and C₂ possibly bearing at least one carbonyl or thiocarbonyl function and being substituted with at least one substituent A₃, or
6) a group C(═NR)R′, C(═NR)NR′R″, COR, CSR, COOR or CONRR′;
c) A₁ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a hydrocarbon-based ring C₆, optionally fused to a ring C₇ optionally containing at least one hetero atom to form a heterocycle Hy₆, these rings C₆ and C₇ optionally bearing at least one carbonyl or thiocarbonyl function and being substituted with at least one substituent A₅,
4) a heterocycle Hy₇ optionally fused to a ring C₇ optionally containing at least one hetero atom to form a heterocycle Hy₆, these rings Hy₇ and C₇ optionally bearing at least one carbonyl or thiocarbonyl function and being substituted with at least one substituent A₅;
d) A₂ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″, or
3) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
e) A₃ represents:
1) A₂, or
2) a C₁-C₂₀ alkyl group optionally substituted with at least one substituent A₅;
f) A₄ represents:
1) a halogen,
2) a group CF₃, CN, OR, SR, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a C₁-C₂₀ alkyl group optionally substituted with at least one substituent A₅,
4) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
g) A₅ represents:
1) a halogen,
2) a group CF₃, CN, OR₅, SR₅, NR₅R₅′, NR₅C(═NR₅′)NR₅″R₅′″, COR₅, CSR₅, COOR₅, CONR₅R₅′, CSNR₅R₅′, NR₅CSR₅′, NR₅CSNR₅′R₅″, NR₅COR₅′, NR₅CONR₅′R₅″, SO₂NR₅R₅′, NR₅SO₂R₅′, SO₂R₅ or SiR₅R₅′R₅″, R₅, R₅′, R₅″ being a hydrogen atom or a C₁-C₂₀ alkyl group,
3) a C₁-C₂₀ alkyl group, or
4) a ring C₁₀ optionally fused to another ring C₁₁, these rings optionally containing at least one hetero atom to form a heterocycle Hy₉ and/or bearing at least one carbonyl or thiocarbonyl function;
h) R, R′, R″ and R′″, which may be identical or different, represent:
1) a hydrogen atom,
2) a C₁-C₂₀ alkyl group optionally substituted with at least one substituent A₅, or
3) a ring C₁₂ optionally fused to another ring C₁₃, these rings optionally containing at least one hetero atom to form a heterocycle Hy₁₀ and being optionally substituted with at least one substituent A₅;
i) Hy₁ to Hy₁₀ independently represent a heterocycle optionally containing from 1 to 4 hetero atoms selected from the group consisting of N, O and S.

6. The regime or regimen as defined by claim 1, wherein formula (I) is selected from the group consisting of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazolidine, dihydopyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine.

7. The regime or regimen as defined by claim 1, wherein formula (I) is selected from the group consisting of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine.

8. The regime or regimen as defined by claim 1, wherein formula (I) is selected from the group consisting of azetidine, dihydropyrrole, pyrrolidine, dihydrofuran, tetrahydrofuran, dihydrothiophene, tetrahydrothiophene, imidazole, dihydroimidazole, imidazolidine, thiazole, dihydrothiazole, thiazolidine, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, diazepine, 2-benzothiazolyl and thiazolo[2,3-c][1,2,4]triazole.

9. The regime or regimen as defined by claim 1, wherein formula (I) is selected from the group consisting of an azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, pyran, dihydropyran, tetrahydropyran, pyrimidine, dihydropyrimidine, tetrahydropyrimidine, piperazine, pyridazine, pyrazine, triazine, morpholine, azepine or diazepine ring, and a crown ether ring containing 15 atoms and 5-CH₂CH₂O— units.

10. The regime or regimen as defined by claim 1, wherein formula (I) Hy₁, Hy₆, Hy₈, Hy₉ and Hy₁₀ are independently selected from the group consisting of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine rings.

11. The regime or regimen as defined by claim 1, wherein the compound of formula (I) has one of the formulae (II) and (III) below: 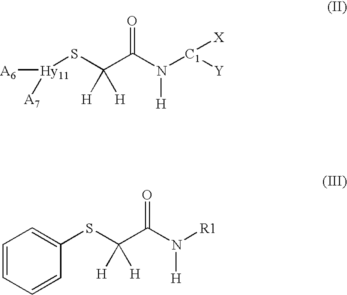
in which X and Y independently represent a hydrogen atom or a halogen atom; C₁ represents a saturated or unsaturated hydrocarbon-based ring of 3 to 6 carbon atoms; Hy₁₁ represents a heterocycle of 5 or 6 atoms containing at least one hetero atom selected from the group consisting of N, S, and a combination thereof; A₆ and A₇ independently represent a substituent selected from the group consisting of hydrogen, alkyl, COR, OR, SR, CN, COOR and saturated or unsaturated rings C₈ of 5 or 6 atoms, optionally comprising from 1 to 4 hetero atoms selected from the group consisting of S, N, and combinations thereof, and/or being optionally fused to a hydrocarbon-based ring C₉ of 5 or 6 carbon atoms; R₁ represents a linear or branched, saturated or unsaturated C₁-C₂₀ alkyl radical, optionally substituted with at least one substituent A₁, a hydrocarbon-based ring or a heterocycle Hy₂ optionally substituted with at least one substituent A₃.

12. The regime or regimen as defined by claim 1, the compound of formula (I) being a salt selected from the group consisting of sodium or potassium salts, zinc (Zn²⁺), calcium (Ca²⁺), copper (Cu²⁺), iron (Fe²⁺), strontium (Sr²⁺), magnesium (Mg²⁺) and manganese (Mn²⁺) salts, triethanolamine, monoethanolamine, diethanolamine, hexadecylamine, N,N,N′,N″-tetrakis(2-hydroxypropyl)ethylenediamine and tris(hydroxymethyl)aminomethane salts, hydroxides and carbonates.

13. The regime or regimen as defined by claim 1, said at least one compound of formula (I) having one of the following formulae:

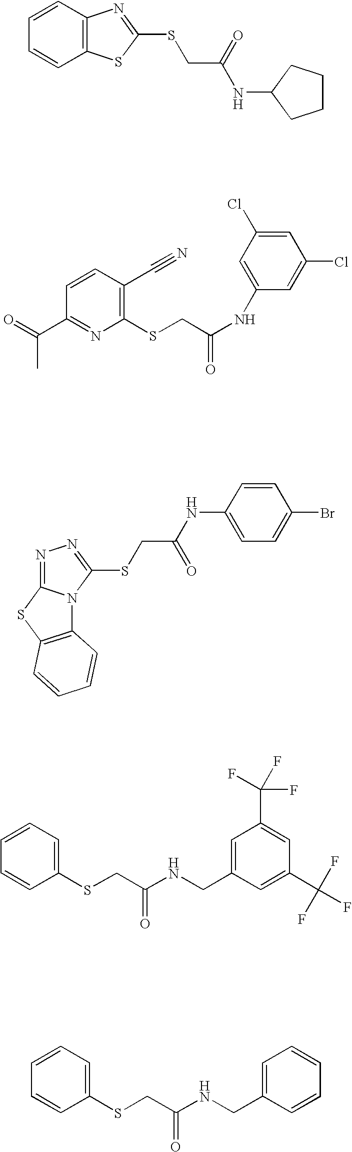

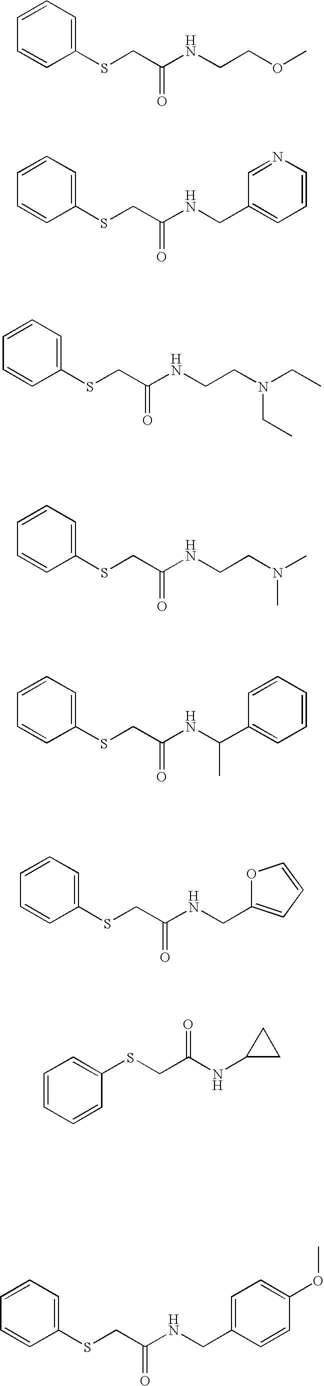

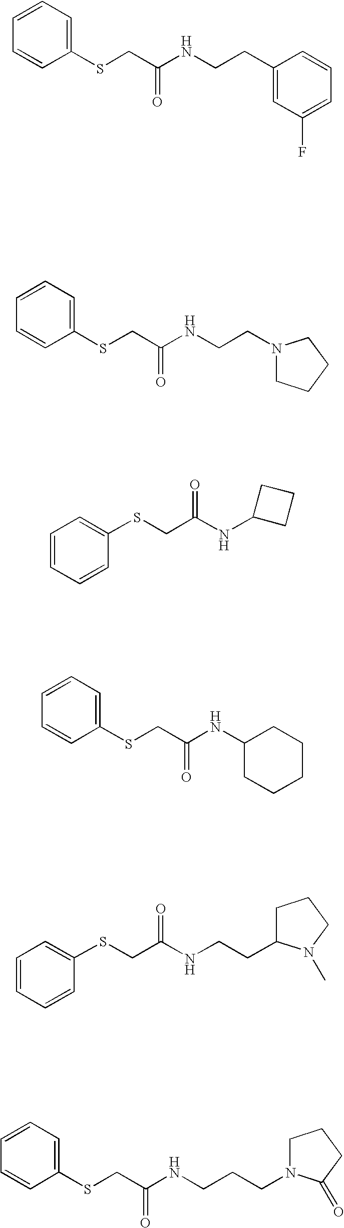

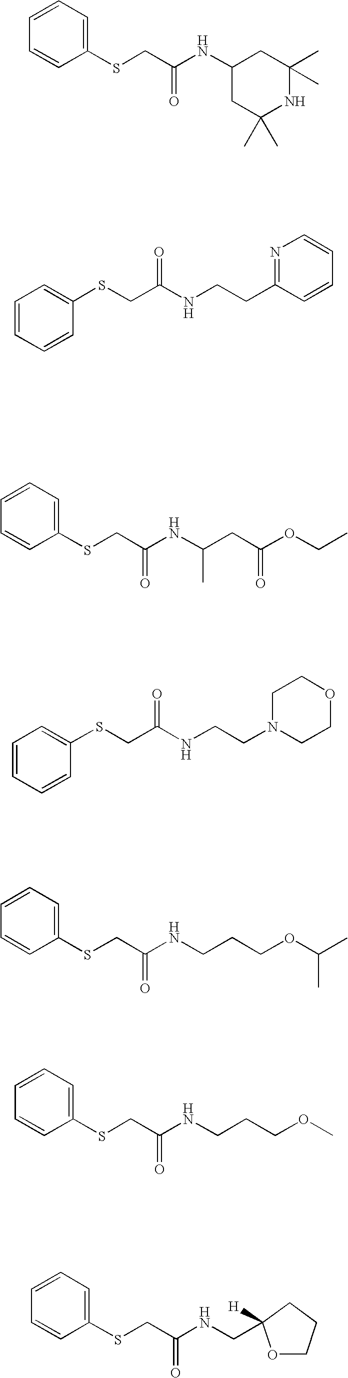

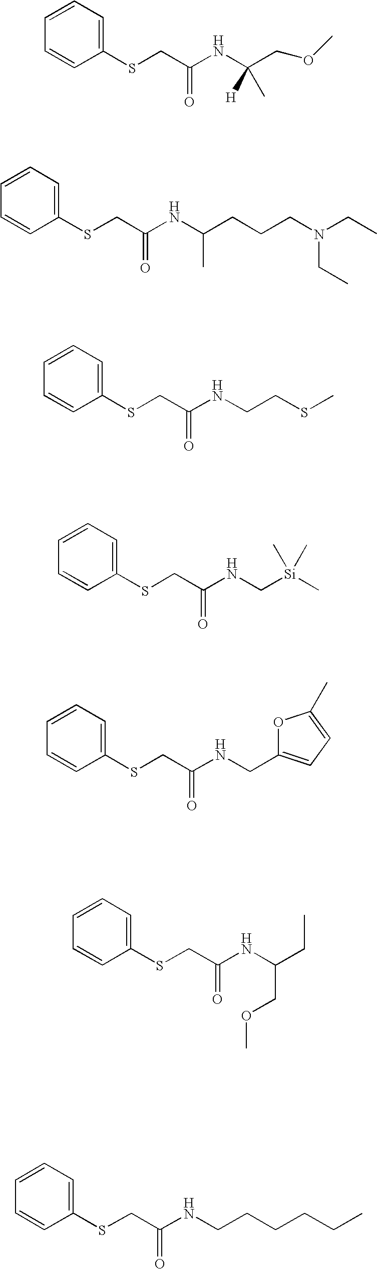

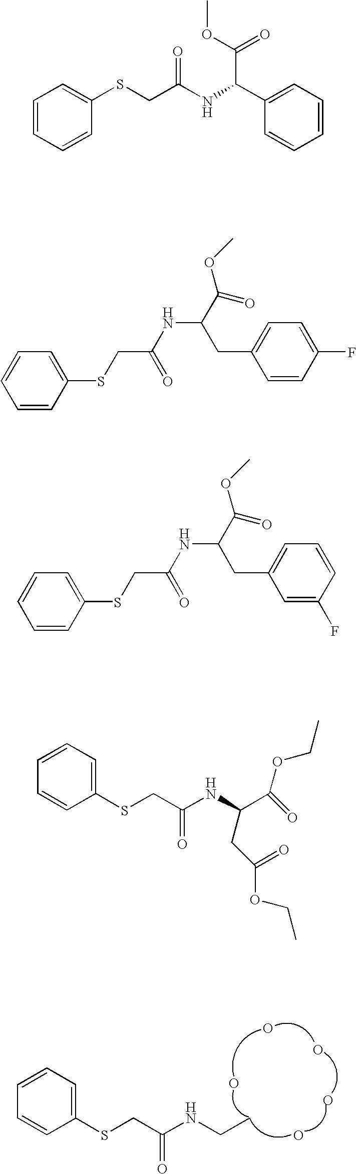

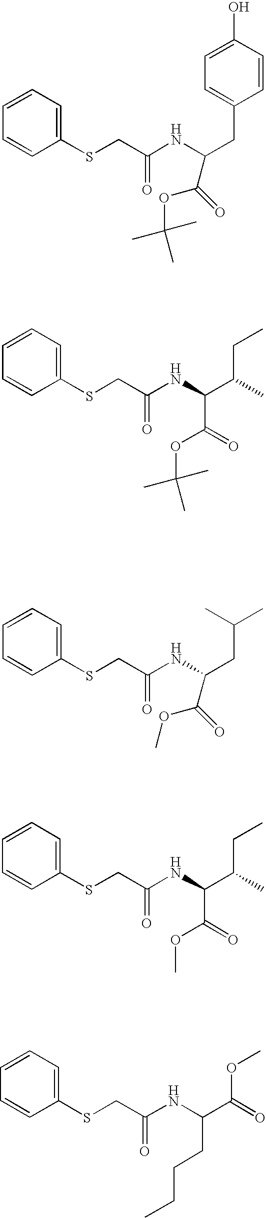

14. A cosmetic composition suited for inducing and/or stimulating the growth of human keratin fibers and/or for reducing the loss and/or increasing the density thereof, comprising a thus-effective amount of at least one 2-thioacetamide compound of formula (I), or salt thereof: 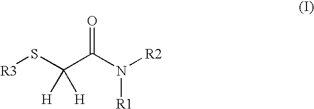
in which:
a) R₁ and R₂ independently represent:
1) a hydrogen atom,
2) a C₁-C₂₀ alkyl radical, optionally substituted with at least one substituent A₁,
3) a hydrocarbon-based ring C₁, optionally fused to a ring C₂ optionally containing at least one hetero atom to form a heterocycle Hy₁, these rings C₁ and C₂ optionally bearing at least one carbonyl or thiocarbonyl function and being substituted with at least one substituent A₃,
4) a heterocycle Hy₂, optionally fused to a ring C₂ optionally containing at least one hetero atom to form a heterocycle Hy₃, these rings Hy₂ and C₂ optionally bearing a carbonyl or thiocarbonyl function and being substituted with at least one substituent A₃, or
5) a group C(═NR)R′, C(═NR)NR′R″, COR, CSR, COOR, CONRR′, SO₂R or SO₂NRR′;
b) R₃ represents:
1) a C₁-C₂₀ alkyl radical, optionally substituted with at least one substituent A₂,
2) a hydrocarbon-based ring C₃, optionally fused to a ring C₄ optionally containing at least one hetero atom to form a heterocycle Hy₃, these rings C₃ and C₄ optionally bearing at least one carbonyl or thiocarbonyl function and being substituted with at least one substituent A₃,
3) a heterocycle Hy₄ representing a pyrrole, furan, thiophene or pyrazole ring, optionally fused to a ring C₅ representing a phenyl, pyridine or pyrimidine ring, these two rings Hy₄ and C₅ optionally being substituted with at least one substituent A₃,
4) a pyridine ring optionally fused to a ring C₂ optionally containing at least one hetero atom to form a heterocycle Hy₁, these pyridine and C₂ rings optionally bearing at least one carbonyl or thiocarbonyl function and being substituted with at least one substituent A₄,
5) a heterocycle Hy₅ other than Hy₄ and the pyridine ring, optionally fused to a ring C₂ optionally containing at least one hetero atom to form a heterocycle Hy₁, these rings Hy₅ and C₂ possibly bearing at least one carbonyl or thiocarbonyl function and being substituted with at least one substituent A₃, or
6) a group C(═NR)R′, C(═NR)NR′R″, COR, CSR, COOR or CONRR′;
c) A₁ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a hydrocarbon-based ring C₆, optionally fused to a ring C₇ optionally containing at least one hetero atom to form a heterocycle Hy₆, these rings C₆ and C₇ optionally bearing at least one carbonyl or thiocarbonyl function and being substituted with at least one substituent A₅,
4) a heterocycle Hy₇ optionally fused to a ring C₇ optionally containing at least one hetero atom to form a heterocycle Hy₆, these rings Hy₇ and C₇ optionally bearing at least one carbonyl or thiocarbonyl function and being substituted with at least one substituent A₅;
d) A₂ represents:
1) a halogen,
2) a group CF₃, CN, OR, SR, NRR′, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″, or
3) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
e) A₃ represents:
1) A₂, or
2) a C₁-C₂₀ alkyl group optionally substituted with at least one substituent A₅;
f) A₄ represents:
1) a halogen,
2) a group CF₃, CN, OR, SR, NRC(═NR′)NR″R′″, COR, CSR, COOR, CONRR′, CSNRR′, NRCSR′, NRCSNR′R″, NRCOR′, NRCONR′R″, SO₂NRR′, NRSO₂R′, SO₂R or SiRR′R″,
3) a C₁-C₂₀ alkyl group optionally substituted with at least one substituent A₅,
4) a ring C₈ optionally fused to a ring C₉, these rings C₈ and C₉ optionally containing at least one hetero atom to form a heterocycle Hy₈ and/or at least one carbonyl or thiocarbonyl function and/or being substituted with at least one substituent A₅;
g) A₅ represents:
1) a halogen,
2) a group CF₃, CN, OR₅, SR₅, NR₅R₅′, NR₅C(═NR₅′)NR₅″R₅′″, COR₅, CSR₅, COOR₅, CONR₅R₅′, CSNR₅R₅′, NR₅CSR₅′, NR₅CSNR₅′R₅″, NR₅COR₅′, NR₅CONR₅′R₅″, SO₂NR₅R₅′, NR₅SO₂R₅′, SO₂R₅ or SiR₅R₅′R₅″, R₅, R₅′, R₅″ being a hydrogen atom or a C₁-C₂₀ alkyl group,
3) a C₁-C₂₀ alkyl group, or
4) a ring C₁₀ optionally fused to another ring C₁₁, these rings optionally containing at least one hetero atom to form a heterocycle Hy₉ and/or bearing at least one carbonyl or thiocarbonyl function;
h) R, R′, R″ and R′″, which may be identical or different, represent:
1) a hydrogen atom,
2) a C₁-C₂₀ alkyl group optionally substituted with at least one substituent A₅, or
3) a ring C₁₂ optionally fused to another ring C₁₃, these rings optionally containing at least one hetero atom to form a heterocycle Hy₁₀ and being optionally substituted with at least one substituent A₅;
i) Hy₁ to Hy₁₀ independently represent a heterocycle optionally containing from 1 to 4 hetero atoms selected from the group consisting of N, O and S, formulated into a physiologically acceptable medium therefor.

15. The cosmetic composition as defined by claim 14, formulated into a topically applicable, physiologically acceptable medium therefor.

16. The cosmetic composition as defined by claim 14, said at least one compound of formula (I) having one of formulae (II) or (III) below: 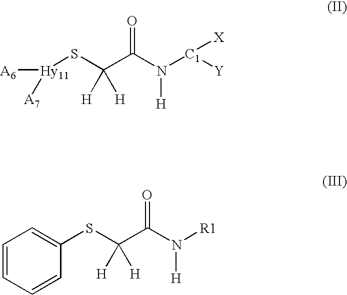
in which X and Y independently represent a hydrogen atom or a halogen atom; C₁ represents a saturated or unsaturated hydrocarbon-based ring of 3 to 6 carbon atoms; Hy₁₁ represents a heterocycle of 5 or 6 atoms containing at least one hetero atom selected from the group consisting of N and S, and a combination thereof; A₆ and A₇ independently represent a substituent selected from the group consisting of hydrogen, alkyl, COR, OR, SR, CN, COOR and saturated or unsaturated rings C₈ of 5 or 6 atoms, optionally comprising from 1 to 4 hetero atoms selected from the group consisting of S and N, and combinations thereof, and/or being optionally fused to a hydrocarbon-based ring C₉ of 5 or 6 carbon atoms; R₁ represents a linear or branched, saturated or unsaturated C₁-C₂₀ alkyl radical, optionally substituted with at least one substituent A₁.

17. The cosmetic composition as defined by claim 14, wherein formula (I), at least one of R₁ and R₂ represents a hydrogen atom; a C₁-C₂₀ alkyl radical, at least one hydrogen of which is replaced with at least one substituent A₁; a saturated or unsaturated hydrocarbon-based ring of 3 to 6 carbon atoms, at least one hydrogen of which is optionally replaced with halogen atoms or CF₃.

18. The cosmetic composition as defined by claim 14, wherein formula (I), R₁ represents H and R₂ represents a cyclopropyl, cyclobutyl, cyclopentyl or cyclohexyl radical or a phenyl radical substituted with a bromine atom or 2 chlorine atoms.

19. The cosmetic composition as defined by claim 14, wherein formula (I), R₁ represents H and R₂ represents a saturated or unsaturated heterocycle of 5 or 6 atoms, containing from 1 to 4 heteroatoms selected from the group consisting of N, O and S and optionally substituted with at least one alkyl, CF₃, OR, SR, NRR′, COR, COOR, CONRR′ or NRCOR′ radical with R and R′ representing H or alkyl.

20. The cosmetic composition as defined by claim 19, wherein formula (I), R₂ represents a piperidine ring optionally substituted with at least one C₁-C₁₀ alkyl radical.

21. The cosmetic composition as defined by claim 14, wherein formula (I), R₁ represents H and R₂ represents a saturated C₁-C₂₀ alkyl, optionally substituted with at least one substituent A₁.

22. The cosmetic composition as defined by claim 21, wherein formula (I), A₁ represents a saturated or unsaturated ring of 5 or 6 atoms, optionally containing from 1 to 4 hetero atoms selected from the group consisting of N, O and S, which may contain at least one carbonyl function and may be optionally substituted with at least one group selected from the group consisting of alkyl, F, CF₃, OR, SR, NRR′, COR, COOR, CONRR′ and NRCOR′ with R and R′ representing H or alkyl, a crown ether ring containing 15 atoms and 5-CH₂CH₂O— units, a group selected from the group consisting of CF₃, OR, SR, NRR′, COR, COOR, CONRR′, NRCOR′ and SiRR′R″ with R, R′ and R″ representing H or alkyl.

23. The cosmetic composition as defined by claim 14, wherein formula (I), R₃ represents a phenyl or a heterocycle of 5 or 6 atoms, substituted with at least one substituent A₃ or fused to a hydrocarbon-based or heterocyclic ring of 5 or 6 atoms.

24. The cosmetic composition as defined by claim 14, wherein formula (I), R₃ represents a phenyl, 2-benzothiazolyl, 2-pyridyl, 6-acetonicotinonitrile, triazolyl or 4a,8a-dihydrobenzo[4,5]thiazolo[2,3-c][1,2,4]triazolyl group.

25. The cosmetic composition as defined by claim 14, said at least one compound of formula (I) being a salt selected from the group consisting of sodium or potassium salts, zinc (Zn²⁺), calcium (Ca²⁺), copper (Cu²⁺), iron (Fe²⁺), strontium (Sr²⁺), magnesium (Mg²⁺) and manganese (Mn²⁺) salts, triethanolamine, monoethanolamine, diethanolamine, hexadecylamine, N,N,N′,N″-tetrakis(2-hydroxypropyl)ethylenediamine and tris(hydroxymethyl)aminomethane salts, hydroxides and carbonates.

26. The cosmetic composition as defined by claim 14, said at least one compound of formula (I) having one of the following formulae:

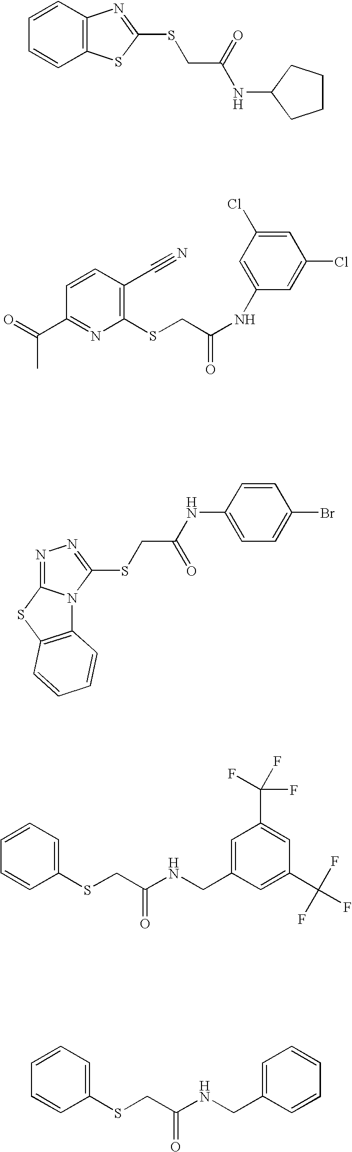

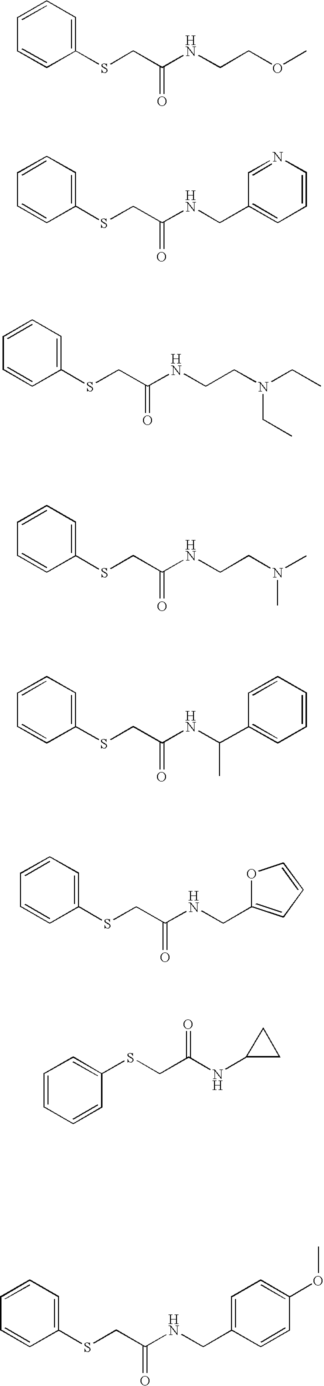

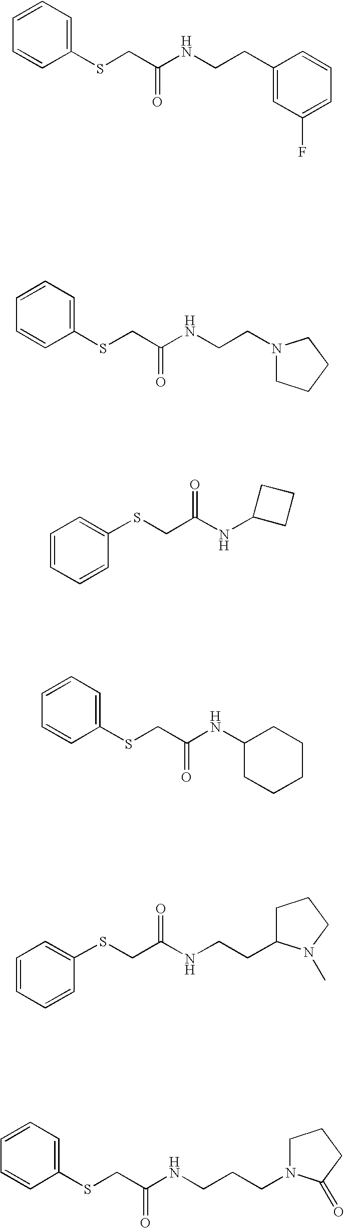

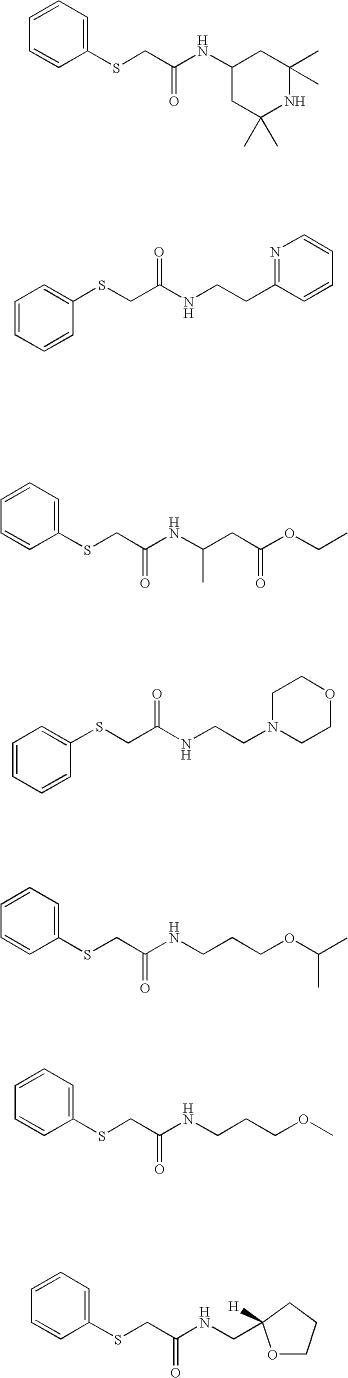

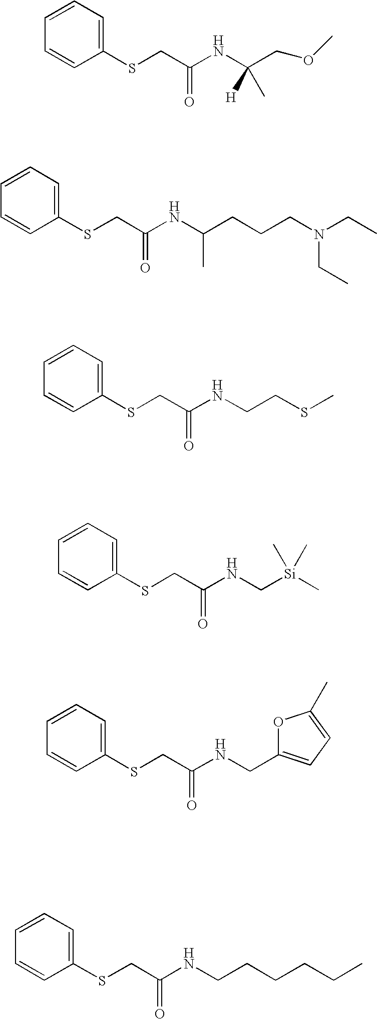

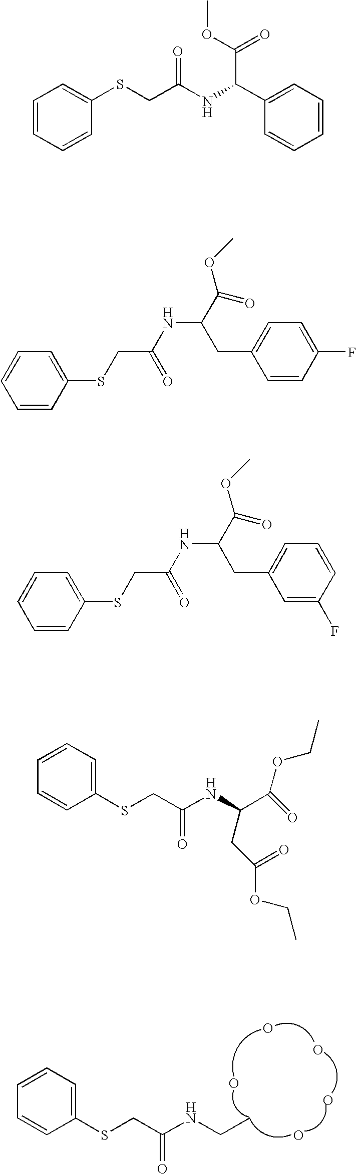

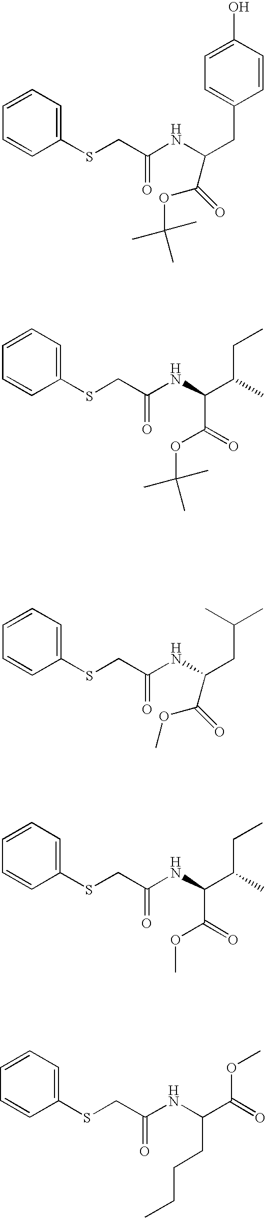

27. The cosmetic composition as defined by claim 14, comprising from 10⁻³% to 5% by weight of said at least one compound of formula (I).

28. The cosmetic composition as defined by claim 14, formulated as a hair cream or hair lotion, a shampoo, a conditioner, or a mascara for the hair or for the eyelashes.

29. The cosmetic composition as defined by claim 14, formulated as an aqueous, alcoholic or aqueous-alcoholic solution or suspension.

30. The cosmetic composition as defined by claim 14, further comprising at least one other ingredient selected from the group consisting of solvents, aqueous-phase or oily-phase thickeners or gelling agents, dyestuffs that are soluble in the medium of the composition, fillers, pigments, antioxidants, preservatives, fragrances, electrolytes, neutralizers, film-forming polymers, UV blockers, cosmetic and pharmaceutical active agents other than the compounds of formula (I), and mixtures thereof.

31. The cosmetic composition as defined by claim 14, further comprising at least one additional active compound that promotes the regrowth and/or limits the loss of keratin fibers.

32. The cosmetic composition as defined by claim 31, said at least one additional active agent being selected from the group consisting of aminexil, 6-0-[(9Z,12Z)octadeca-9,12-dienoyl]hexapyranose, lipoxygenase inhibitors, bradykinin inhibitors, prostaglandins and derivatives thereof, prostaglandin receptor agonists or antagonists, non-prostanoic prostaglandin analogues, vasodilators, antiandrogens, cyclosporins and analogues thereof, antimicrobial agents, anti-inflammatory agents, retinoids, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharide or acylhexosaccharide acids, substituted aryl ethylenes, N-acylamino acids, flavonoids, ascomycin derivatives and analogues, histamine antagonists, saponins, proteoglycanase inhibitors, oestrogen agonists and antagonists, pseudoterines, cytokines, growth factor promoters, IL-1 or IL-6 inhibitors, IL-10 promoters, TNF inhibitors, vitamins, benzophenones, hydantoin, retinoic acid, antipruriginous agents, antiparasitic agents, antifungal agents, calcium antagonists, hormones, triterpenes, antiandrogenic agents, steroidal or non-steroidal 5-α-reductase inhibitors, potassium channel agonists and FP receptor antagonists, and mixtures thereof.

33. The cosmetic composition as defined by claim 14, further comprising at least one active agent selected from the group consisting of proteins or protein hydrolysates, amino acids, polyols, urea, allantoin, sugars and sugar derivatives, water-soluble vitamins, plant extracts, hydroxy acids, retinol, tocopherol, retinol or tocopherol derivatives, essential fatty acids, ceramides, essential oils, salicylic acid derivatives, for instance 5-n-octanoylsalicylic acid, hydroxy acid esters, and phospholipids, and mixtures thereof.

34. The cosmetic composition as defined by claim 14, further comprising at least one additional active agent that promotes the regrowth of human keratin fibers and/or that limits the loss thereof, selected from the group consisting of aminexil, FP receptor agonists and vasodilators.

35. The cosmetic composition as defined by claim 34, said at least one additional active agent being selected from the group consisting of aminexil, minoxidil, latanoprost and travoprost.
